SEQUENCE LISTING (69 sequences)

SEQ ID NO: 1 - DNA, 18 bp, Artificial Sequence
  PCR primer - T7
taatacgactcactatag

SEQ ID NO: 2 - DNA, 18 bp, Artificial Sequence
  PCR primer - T3
aattaaccctcactaaag

SEQ ID NO: 3 - DNA, 20 bp, Artificial Sequence
  PCR primer - HF1
gaygartgyccnmgnatntg

SEQ ID NO: 4 - DNA, 18 bp, Artificial Sequence
  PCR primer - HF2
gartgyccnmgnatntgy

SEQ ID NO: 5 - DNA, 17 bp, Artificial Sequence
  PCR primer - HF3
acnttyggnaaycartg

SEQ ID NO: 6 - DNA, 20 bp, Artificial Sequence
  PCR primer - HR1
aatacaacatattcaagtgg

SEQ ID NO: 7 - DNA, 31 bp, Artificial Sequence
  PCR primer - HF6
gtacggatccatgaaatttgccttgttcagt

SEQ ID NO: 8 - DNA, 52 bp, Artificial Sequence
  PCR primer - HR3
catgctgcagttagtgatggtgatggtgatgacccttgcactcgccatcatg

SEQ ID NO: 9 - DNA, 19 bp, Artificial Sequence
  Primer - PFBR
gattatgatcctctagtac

SEQ ID NO: 10 - DNA, 20 bp, Artificial Sequence
  Primer - PFBF
tattccggattattcatacc

SEQ ID NO: 11 - protein, 76 aa, Hybomitra bimaculata
  SIGNAL 1-20
MKFALFSVLVVLLIATFVAADECPRICTADYRPVCGTPSGGRRSANRTFGNQCSLNAHNCLNKGDTYDKLHDGECK

SEQ ID NO: 12 - DNA, 331 bp, Hybomitra bimaculata
  CDS 56-285
  misc_feature 48: n = a, c, g, or t
gtttagttcagtttttatagtaaccagttctaaaagtttaataacatnaatcaaaatgaaatttgccttgttcagtgttttagttgttctgctgattgcaacatttgttgcggctgatgaatgcccacgtatttgcacggctgactatagaccggtatgcggcactccctctggtggtcgccgaagtgcaaacaggacttttggaaaccaatgtagcctcaacgcccacaactgcttgaacaagggagatacttacgacaaactgcatgatggcgagtgcaagtaaaaaggacaagtcccaggaatattattgactccacttgaatatgta

SEQ ID NO: 13 - protein, 61 aa, Artificial Sequence
  Kazal-type inhibitor consensus
CSRYPNPTSKDGKLVACPREYDPVCGSDGVTYSNECELKKAACAENVEQGTNIEKKHDGPC

SEQ ID NO: 14 - protein, 7 aa, Hybomitra bimaculata
PSGGRRS

SEQ ID NO: 15 - protein, 43 aa, Rhodnius prolixus
CACPHALHRVCGSDGETYSNPCTLNVAKFGKEPELVKVHDGPC

SEQ ID NO: 16 - protein, 45 aa, Rhodnius prolixus
CQECDGDEYKPVCGSDDITYDNNCRLECASISSSPGVELKHEGPC

SEQ ID NO: 17 - protein, 45 aa, Anemonia sulcata
CPLICTMQYDPVCGSDGITYGNACMLLGASCRSDTPIELVHKGRC

SEQ ID NO: 18 - protein, 46 aa, Gallus gallus
CKKTACPVVVAPVCGSDYSTYSNECELEKAQCNQQRRIKVISKGPC

SEQ ID NO: 19 - protein, 49 aa, Homo sapiens
CSQYRLPGCPRHFNPVCGSDMSTYANECTLCMKIREGGHNIKIIRNGPC

SEQ ID NO: 20 - protein, 45 aa, Gallus gallus
CDFTCLAVPRSPVCGSDDVTYANECELKKTRCEKRQNLVTSQGAC

SEQ ID NO: 21 - protein, 46 aa, Rattus norvegicus
CDFSCQSVPRSPVCGSDGVTYGTECDLKKARCESQQELYVAAQGAC

SEQ ID NO: 22 - protein, 47 aa, Homo sapiens
CAPDCSNITWKGPVCGLDGKTYRNECALLKARCKEQPELEVQYQGRC

SEQ ID NO: 23 - protein, 46 aa, Gallus gallus
CPASCSGVAESIVCGSDGKDYRSECDLNKHACDKQENVFKKFDGAC

SEQ ID NO: 24 - protein, 46 aa, Rattus norvegicus
CPTTCFGAPDGTVCGSDGVDYPSECQLLSHACASQEHIFKKFNGPC

SEQ ID NO: 25 - protein, 45 aa, Gallus gallus
CQQVCQGRYDPVCGSDNRTYGNPCELNAMACVLKREIRVKHKGPC

SEQ ID NO: 26 - protein, 45 aa, Rattus norvegicus
CQRVCAGIYDPVCGSDGVTYGSVCELESMACTLGREIQVARRGPC

SEQ ID NO: 27 - protein, 49 aa, Rattus norvegicus
CEHMTESPDCSRIYDPVCGTDGTYESECKLCLARIENKQDIQIVKDGEC

SEQ ID NO: 28 - protein, 48 aa, Rattus norvegicus
CPKQIMGCPRIYDPVCGTNGITYPSECSLCFENRKFGTSIHIQRRGTC

SEQ ID NO: 29 - protein, 48 aa, Rattus norvegicus
CPNTLVGCPRDYDPVCGTDGKTYANECILCFENRKFGTSIRIQRRGLC

SEQ ID NO: 30 - protein, 48 aa, Bos taurus
CTNEVNGCPRIYNPVCGTDGVTYSNECLLCMENKERQTPVLIQKSGPC

SEQ ID NO: 31 - protein, 47 aa, Canis familiaris
CNLKVNGCNKIYNPICGSDGITYANCLLCLENKKRQTSILVEKSGPC

SEQ ID NO: 32 - protein, 45 aa, Gallus gallus
CPTECVPSSQPVCGTDGNTYGSECELHVRACTQQKNILVAAQGDC

SEQ ID NO: 33 - protein, 45 aa, Rattus norvegicus
CPSECVESAQPVCGSDGHTYASECELHVHACTHQISLYVASAGHC

SEQ ID NO: 34 - protein, 45 aa, Gallus gallus
CPRCEQQPLAQVCGTDGLTYDNRCELRAASCQQQKSIEVAKMGPC

SEQ ID NO: 35 - protein, 45 aa, Rattus norvegicus
CPRCEHPPPGPVCGSDGVTYLSACELREAACQQQVQIEEAHAGPC

SEQ ID NO: 36 - protein, 47 aa, Gallus gallus
CPSPLCSEANMTKVCGSDGVTYGDQCQLKTIACRQGQLITVKHVGQC

SEQ ID NO: 37 - protein, 47 aa, Rattus norvegicus
CPTLTCPEANSTKVCGSDGVTYGNECQLKAIACRQRLDISTQSLGPC

SEQ ID NO: 38 - protein, 58 aa, Gallus gallus
CSLYASGIGKDGTSWVACPRNLKPVCGTDGSTYSNECGICLYNREHGANVEKEYDGEC

SEQ ID NO: 39 - protein, 57 aa, Gallus gallus
CSPYLQVVRDGNTMVACPRILKPVCGSDSFTYDNECGICAYNAEHHTNISKLHDGEC

SEQ ID NO: 40 - protein, 58 aa, Gallus gallus
CSKYPSTVSKDGRTLVACPRILSPVCGTDGFTYDNECGICAHNAEQRTHVSKKHDGKC

SEQ ID NO: 41 - protein, 58 aa, Gallus gallus
CDQYPTRKTTGGKLLVRCPRILLPVCGTDGFTYDNECGICAHNAQHGTEVKKSHDGRC

SEQ ID NO: 42 - protein, 58 aa, Gallus gallus
CSRFPNATDKEGKDVLVCNKDLRPICGTDGVTYTNDCLLCAYSIEFGTNISKEHDGEC

SEQ ID NO: 43 - protein, 58 aa, Coturnix coturnix
CSRFPNTTNEEGKDEVVCPDELRLICGTDGVTYNHECMLCFYNKEYGTNISKEQDGEC

SEQ ID NO: 44 - protein, 58 aa, Gallus gallus
CSSYANTTSEDGKVMVLCNRAFNPVCGTDGVTYDNECLLCAHKVEQGASVDKRHDGGC

SEQ ID NO: 45 - protein, 58 aa, Coturnix coturnix
CSRYPNTTSEDGKVTILCTKDFSFVCGTDGVTYDNECMLCAHNVVQGTSVGKKHDGEC

SEQ ID NO: 46 - protein, 58 aa, Gallus gallus
CSKYKTSTLKDGRQVVACTMIYDPVCATNGVTYASECTLCAHNLEQRTNLGKRKNGRC

SEQ ID NO: 47 - protein, 50 aa, Anguilla anguilla
CGEMSAMHACPMNFAPVCGTDGNTYPNECSLCFQRQNTKTDILITKDDRC

SEQ ID NO: 48 - protein, 46 aa, Gallus gallus
CDRITCDGTYRPVCARDSRTYSNDCERQKAECHQKAAIPVKHSGPC

SEQ ID NO: 49 - protein, 46 aa, Rattus norvegicus
CDRVTCDGSYRPVCAQDGHTYNNDCWRQQAECRQQRAIPPKHQGPC

SEQ ID NO: 50 - protein, 47 aa, Homo sapiens
CDELCPDSKSDEPVCASDNATYASECAMKEAACSSGVLLEVKHSGSC

SEQ ID NO: 51 - protein, 53 aa, Canis familiaris
CSNYKGKGSQIACPRLHQPICGTDHKTYSNECMFCALTLNKKFEVRKLQDTAC

SEQ ID NO: 52 - protein, 53 aa, Meles meles
CSKYNAKGSQFACSRHLDPVCGTDHRTYSNECMFCMLTQNKRFSVRILQDNNC

SEQ ID NO: 53 - protein, 53 aa, Felis silvestris catus
CSQYNRKGSGITCSKEWKPICGIDHKTYSNECMFCQLNQNKRFQLRKLHDNKC

SEQ ID NO: 54 - protein, 48 aa, Bos taurus
CKVYTEACTREYNPICDSAAKTYSNECTFCNEKMNNDADIHFNHFGEC

SEQ ID NO: 55 - protein, 51 aa, Sus scrofa
CNVYRSHLFFCTRQMDPICGTNGKSYANPCIFCSEKGLRNQKFDFGHWGHC

SEQ ID NO: 56 - protein, 51 aa, Bos taurus
CAEFKDPKVYCTRESNPHCGSNGETYGNKCAFCKAVMKSGGKINLKHRGKC

SEQ ID NO: 57 - protein, 51 aa, Gallus gallus
CREFQKVSPICTMEYVPHCGSDGVTYSNRCFFCNAYVQSNRTLNLVSMAAC

SEQ ID NO: 58 - protein, 49 aa, Aburria pipile
CSDHPKPACLQEQKPLCGSDNKTYDNKCSFCNAVVDSNGTLTLSGFGKC

SEQ ID NO: 59 - protein, 49 aa, Coqui francolin
CSEYPKPGCTMEYRPVCGSDNITYGNKCNFCNAVVKSNGTLTLSHFGKC

SEQ ID NO: 60 - protein, 49 aa, Casuarius casuarius
CSEYPKPVCSPEYMPLCGSDSKTYNNKCDFCSAVVESNGTLTLGHFGKC

SEQ ID NO: 61 - protein, 47 aa, Eudromia elegans
CSGYPKPACTLEFFPLCGSDNQTYSNKCAFCNAAVEKNVTLNHIGEC

SEQ ID NO: 62 - protein, 48 aa, Canis familiaris
CTEYSDMCTMDYRPLCGSDGKNYSNKCSFCNAVKKSRGTIFLAKHGEC

SEQ ID NO: 63 - protein, 48 aa, Felis silvestris catus
CTNYSAICTMEYFPLCGSDGQEYSNKCLFCNEVVKRRGTLFLAKYGQC

SEQ ID NO: 64 - protein, 47 aa, Gallus gallus
LSRPENCPSKREPVCGDDGVTYASECVMGRTGAIRGLEIQKVRSGQC

SEQ ID NO: 65 - protein, 48 aa, Rattus norvegicus
MLLRPENCPAQHTPICGDDGVTYENDCVMSRIGAREGLLLQKVRSGQC

SEQ ID NO: 66 - protein, 48 aa, Homo sapiens
CNRICPEPASSEQYLCGNDGVTYSSACHLRKATCLLGRSIGLAYEGKC

SEQ ID NO: 67 - protein, 54 aa, Coturnix coturnix
CQDPAACPSTKDYKRVCGTDNKTYDGTCQLFGTKCQLEGTKMGRQLHLDYMGAC

SEQ ID NO: 68 - protein, 54 aa, Rattus norvegicus
CQDPETCPPAKILDQACGTDNQTYASSCHLFATKCMLEGTKKGHQLQLDYFGAC

SEQ ID NO: 69 - protein, 55 aa, Bos taurus
CQDPTSCPAPIGEFEKVCSNDNKTFDSSCHFFATKCTLEGTKKGHKLHLDYIGPC

(19) United States
(12) Patent Application Publication
(10) Pub. No.: US 2005/0090437 A1
(43) Pub. Date: Apr. 28, 2005

(54) Ion channel modulators

(76) Inventors: Patricia Anne Nuttall, Abingdon (GB); Miles Andrew Nunn, Oxford (GB); Peter Takac, Dunajska Luzna (SK); Olga Pechanova, Bratislava (SK); Petra Rajska, Bratislava (SK); Milan Kozanek, Bratislava (SK); Norbert Vrbjar, Bratislava (SK); Maria Kazimirova, Dunajska Luzna (SK); Milan Labuda, Borinka (SK)

Correspondence Address: KLAUBER & JACKSON, 411 HACKENSACK AVENUE, HACKENSACK, NJ, 07601, US

(21) Appl. No.: 10/743,280

(22) Filed: Dec. 22, 2003

Related U.S. Application Data
(63) Continuation of application No. PCT/GB02/02919, filed on Jun. 21, 2002.

(30) Foreign Application Priority Data
Jun. 22, 2001 (GB) 0115363.4

(51) Int. Cl. A61K 38/16; A01N 65/00; C07H 21/04; C12N 5/06; C07K 14/435

(52) U.S. Cl. 514/12; 435/69.1; 435/320.1; 435/348; 530/350; 536/23.5

(57) ABSTRACT

The present invention relates to ion channel modulators. In particular, the invention relates to ion channel modulators that are derived from haematophagous arthropods. The invention also relates to the use of ion channel modulators from haematophagous arthropods in the treatment and prevention of certain diseases and conditions in mammals, including humans.

### RELATED APPLICATIONS

The present application is a Continuation of co-pending PCT Application No. PCT/GB02/02919 filed on Jun. 21, 2002, which in turn, claims priority from Great Britain Application Serial No. 0115363.4, filed Jun. 22, 2001. Applicants claim the benefits of 35 U.S.C. §120 as to the PCT application and priority under 35 U.S.C. §119 as to said Spanish application, and the entire disclosures of both applications are incorporated herein by reference in their entireties.

The present invention relates to ion channel modulators. In particular, the invention relates to ion channel modulators that are derived from haematophagous arthropods. The invention also relates to the use of ion channel modulators from haematophagous arthropods in the treatment and prevention of certain diseases and conditions in mammals, including humans.

Ion channels are proteins which allow ions to cross the lipid bilayer of cell membranes. They exert control over the movement of ions by being either open or closed. Ion channel proteins can be divided into two groups. The first group of ion channel proteins form narrow hydrophilic pores across the membrane, allowing the passive movement of small inorganic ions. The second group of ion channel proteins can be coupled to a source of energy to promote active transport of ions across the cell membrane. Through a combination of passive and active transport using ion channels, cells can generate ionic concentration differences across the lipid bilayer of cell membranes.

The electrical gradient thus generated can be used by cells in their signalling and control systems. Ion channels show selectivity with respect to the ions to which they are permeable and respond to different opening and closing stimuli (eg ligand gated or voltage gated channels). Modulators of ion channels can be divided into blocking agents which can only close channels and modulators which can either open or close them (Hille, 1992).

The present invention is particularly concerned with the modulation of ion channels in vascular smooth muscle cells and cardiac muscle cells. In such cells, propagation of an action potential over the membrane surface of the cell is associated with contraction of the cell. This effect is usually achieved by a small change in the resting membrane potential triggering the opening of a voltage gated ion channel which then permits the influx of large quantities of positively charged ions with a consequent change in the electrostatic conformation of the cell. Typically, the small change in the resting membrane potential is achieved through active transport of Na⁺ and K⁺ ions by the Na⁺−K⁺ pump. This pump depends on an energy source to drive the process and this is provided by the catalysis of ATP by the enzyme Na,K-ATPase.

The contraction of smooth muscle, including cardiac smooth muscle, is dependent on a transient increase in cytoplasmic Ca⁺⁺ concentration, such ions coming from two sources: influx from the extracellular compartment by opening of inward Ca⁺⁺ channels and release of intracellular Ca⁺⁺ ions from the cisternae of the sarcoplasmic reticulum. Calcium channel blocking agents that inhibit the influx of extracellular Ca⁺⁺ ions will therefore tend to relax most smooth muscle cells including cardiac cells. The force of contraction of cardiac smooth muscle is termed the inotropic state and, therefore, agents which relax the resting state of cardiac smooth muscle cells, such as most calcium channel blockers, are negatively inotropic. Conversely agents that increase the force of contraction of cardiac smooth muscle are positively inotropic.

Calcium channel blockers are used therapeutically to relax vascular smooth muscle thereby increasing the diameter of blood vessels (‘vasodilation’) in order to lower blood pressure or increase regional blood flow (eg via the coronary arteries). The coincidental lowering of the force of cardiac smooth muscle contraction is an inherent disadvantage of most calcium channel blocking agents and precludes their use in conditions in which the functioning of the heart is already compromised. Such conditions include congestive heart failure, cardiomyopathies, septicaemia and following myocardial infarction. (Alberts et al, 1998; Camm, 1996a; Hume et al, 1998; Akera et al, 1998).

There is therefore a need for agents which possess the desirable qualities of vasodilators but which are not negatively inotropic. Preferably, such agents would be positively inotropic.

In situations in which it is desired to increase cardiac output, drugs having a positive inotropic effect may be used. Positively inotropic drugs such as digoxin frequently work through inhibition of sodium-potassium ATPase (Camm, 1996b). However, currently known positively inotropic drugs, which include digoxin, dopamine, isopraline and phopshodiesterase inhibitors such as enoximone and milrinone, often have other, undesirable, effects on the heart such as the induction of arrhythmia.

There is therefore a need for agents which have a positive intropic effect on the heart without inducing arrythmias.

Given the importance of ion channels in disease, there is thus a need for the identification of novel ion channel modulators with improved properties. More specifically, in view of the widespread incidence of cardiovascular disease and the disadvantages of drugs that are currently available, there remains a need for improved ion channel modulators in the treatment of cardiovascular disease. In particular, there remains a great need for ion channel modulators with properties which are useful in the treatment of cardiovascular disease, such as those that induce vasodilation and that are positively inotropic. In addition, there is a need for ion channel modulators which can induce a positive inotropic effect without having adverse effects, such as inducing arrhythmia.

It has now been discovered that suitable ion channel modulators can be isolated from haematophagous arthropods.

### SUMMARY OF THE INVENTION

According to a first aspect of the present invention, there is provided an ion channel modulator molecule (ICMM), derived from an haematophagous arthropod, or a functional equivalent thereof.

By “ion channel modulator molecule” is meant any molecule that modulates the activity of an ion channel. Preferably, the ICMMs are proteins or peptides. However, they may also be non-peptidic derivatives. Preferably, non-peptidic derivatives are small organic molecules.

By “ion channel” is meant any transmembrane protein or transmembrane protein complex present in the cell that allows the movement of particular ions from one side of a cell membrane to the other.

The ICMMs or functional equivalents of the present invention modulate the activity of ion channels that allow the movement of ions across the cell membrane by either active or passive transport. Examples of ion channels that allow the movement of ions by active transport include calcium ATPase and sodium-potassium ATPase. Examples of ion channels that allow movement of ions by passive transport include calcium channels, sodium channels and potassium channels. These channels tend to be of particular relevance to disease, such as cardiac disease.

The ICMMs or functional equivalents of the invention may modulate more than one ion channel or class of ion channel. Such modulators may allow the simultaneous modulation of ion channels of different classes. For example, ICMMs may permit vasodilation by means of blockade of one or more calcium channels whilst also causing positive inotropism by means of inhibition of the sodium-potassium ATPase channel.

The ICMMs or functional equivalents of the present invention may modulate the activity of ion channels by either inhibiting or promoting the activity of ion channels. Preferably, the ICMMs or functional equivalents of the invention inhibit the activity of ion channels. One example of an ion channel that the ICMMs or functional equivalents of the present invention may inhibit is the sodium-potassium ATPase ion channel. Preferably, ICMMs or functional equivalents of the invention inhibit the activity of ion channels by binding to them.

The ICMMs or functional equivalents may be vasodilators. Vasodilation may be promoted by the ICMMs functional equivalents of the invention, for example, through blockade of calcium channels or by nitric oxide donation. The ICMMs or functional equivalents may be vasodilators of coronary vessels, peripheral vessels or both types of vessel. Preferably, the ICMMs, or functional equivalents of the invention act as vasodilators of both coronary vessels and peripheral vessels.

The ICMMs or functional equivalents of the invention preferably do not induce a negative inotropic effect in cardiac smooth muscle. Preferably, the ICMMs or functional equivalents induce a positive inotropic effect.

The ICMMs or functional equivalents of the invention may function to prolong the action potential of muscle cells. Preferably, the ICMMs or functional equivalents of the invention prolong the action potential of cardiomyocyte cells. This property may be important in prevention or treatment of arrhythmias such as are sometimes associated with the use of other positively inotropic drugs.

The ICMMs or functional equivalents of the present invention are derived from haematophagous arthropods. The term “haematophagous arthropod” includes all arthropods that take a blood meal from a suitable host, and includes insects, ticks, lice, fleas and mites.

Preferably, the ICMMs or functional equivalents of the present invention are derived from horseflies of the Tabanidae family. More preferably, they are derived from horseflies of the Hybomitra, Heptatoma, Chrysops, Haematopota and Tabanus genera. Most preferably, they are derived from the horsefly Hybomitra bimaculata.

The term “functional equivalent” is used herein to describe variants, derivatives or fragments of ICMMs of the invention that retain the ability to modulate ion channels. Functional equivalents of the ICMMs of the present invention thus include natural biological variants (e.g. allelic variants or geographical variants within the species from which the ICMMs are derived).

Variants of the proteinaceous ICMMs of the invention also include, for example, mutants containing amino acid substitutions, insertions or deletions from the wild type sequence. Variants with improved function from that of the wild type sequence may also be designed through the systematic or directed mutation of specific residues in the protein sequence. Improvements in function that may be desired will include greater specificity for the target ion channel or greater affinity for the target ion channel.

The term “functional equivalent” also refers to molecules that are structurally similar to the proteinaceous ICMMs of the present invention or that contain similar or identical tertiary structure, particularly in the environment of the active site. Such functional equivalents may thus be derived from natural proteinaceous ICMMs or they may be prepared synthetically or using techniques of genetic engineering. In particular, synthetic molecules that are designed to mimic the tertiary structure or active site of the natural proteinaceous ICMMs of the invention are considered to be functional equivalents.

The term “functional equivalent” also includes fragments of the proteinaceous ICMMs of the present invention, fragments of variants of the proteinaceous ICMMs and fragments of structurally similar molecules, provided such fragments retain the ability to modulate ion channels. Preferably, protein fragments according to the invention comprise the amino acid sequence psggrrs. This amino acid sequence may be the active site of proteinaceous ICMMs, although the Applicant does not wish to be bound by this theory.

The term “functional equivalent” also refers to homologues of the proteinaceous ICMMs. By “homologue” is meant a protein exhibiting a high degree of similarity or identity to the amino acid sequence of a natural proteinaceous ICMM. By “similarity” is meant that, at any particular position in the aligned sequences, the amino acid residue is of a similar type between the sequences. By “identity” is meant that at any particular position in the aligned sequences, the amino acid residue is identical between the sequences.

Preferably, homologues possess greater than 50% identity with the sequence of the natural protein. More preferably, homologues according to the invention show greater than 60%, 70%, 80%, 85%, 90%, 95%, 97%, 98% or 99% sequence identity with the sequence of the natural protein, as aligned using, for example, the GCG suite of programs (Wisconsin Package Version, 10.1, Genetics Computer Group (GCG), Madison, Wis.) or the ExPASy (ExpertProtein Analysis System) proteomics server of the Swiss Insitute of Bioinformatics. Tools such as PROSITE (http://expasy.hcuge.ch/sprot/prosite.html), PRINTS http://iupab.leeds.ac.uk/bmb5dp/prints.html), Profiles (http://ulrec3.unil.ch/software/PFSCAN_form.html), Pfam (http://www.sanger.ac.uk/software/pfam), Identify (http://dna.stanford.edu/identify/) and Blocks (http://www.blocks.fhcrc.org) databases may also be used to identify homologues, as well as hidden Markov models (HMMs; preferably profile HMMs). Such homologues may include proteins in which one or more of the amino acid residues are substituted with another amino acid residue and such substituted amino acid residue may or may not be a naturally occurring amino acid.

The term “functional equivalent” also includes derivatives of the present invention. Such derivatives may include one or more additional peptides or polypeptides fused at the amino- or carboxy-terminus of the proteinaceous ICMMs, fragments or homologues. The purpose of the additional peptide or polypeptide may be to aid the detection, expression, separation or purification of the protein or it may endow the protein with additional properties, as desired. Examples of useful fusion partners include beta-galactosidase, glutathione-S-transferase, luciferase, a polyhistidine tag, a T7 polymerase fragment and a secretion signal peptide. Such derivatives may be prepared by fusing the peptides genetically or chemically.

The proteinaceous ICMMs or functional equivalents of the present invention may show some homology to Kazal type proteins. The Kazal family of proteins includes a variety of protease inhibitors including pancreatic secretory trypsin inhibitor (Greene and Giordano, 1969), avian ovomucoid (Laskowski et al, 1987), acrosin inhibitor (Williamson et al, 1984) and elastase inhibitor (Tschesche et al. 1987). The basic structure of a Kazal-type inhibitor is shown in the following schematic representation: 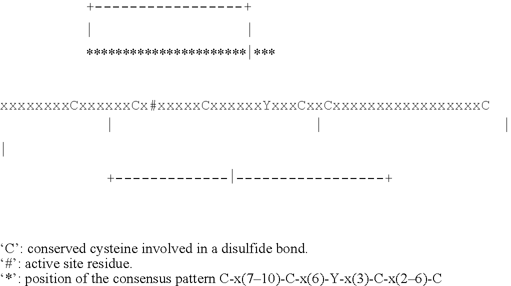
Kazal inhibitors contain between 1 and 9 Kazal-type inhibitor repeats.
The Applicant has demonstrated that at least one proteinaceous ICMM shows homology with Kazal type proteins and believes that other Kazal type proteins may act as ICCMs.

A further embodiment of the invention therefore provides the use of a Kazal type protein, or functional equivalent thereof as an ICMM. Functional equivalents of Kazal type proteins include fragments and variants of Kazal type proteins, providing that said fragments and variants retain ion channel modulatory activity. In particular, the term functional equivalent is used to describe variants of Kazal proteins obtained by mutation or trunction of a Kazal type protein.

According to a preferred embodiment of the invention, the ICMM comprises the amino acid sequence set out in FIG. 9a, or a functional equivalent thereof. The ICMM comprising this sequence is referred to herein as EV048. Functional equivalents of EV048 include variants, fragments, homologues or derivatives as defined above, providing that said variants, fragments homologues or derivatives retain activity as ion channel modulators. Amino acids 1 to 20 of the EV048 amino acid sequence set out in FIG. 9a form a signal sequence. Functional equivalents of EV048 include fragments of the amino acid sequence set out in FIG. 9a which do not contain the signal sequence.

Preferably, variants, fragments, homologues or derivatives of EV048 should retain structural homology or conservation of the putative active site of EV048. This active site may reside in the 7 amino acid sequence psggrrs between the third and fourth cysteine molecules in the sequence, although the Applicant does not wish to be bound by this theory.

According to a further embodiment of the invention, there is provided the use of a peptide or polypeptide comprising the sequence psggrrs as an ion channel modulator, as defined above.

The invention also provides a screening method for the identification of variants of EV048 which have enhanced ion modulatory properties compared to EV048. According to this method, variants of EV048, preferably mutants, are created and these mutants are tested for the ability to modulate ion channel activity. In particular, such mutants are tested for their ability to cause vasodilation and positive inotropism.

The ICMMs or functional equivalents of the invention may be prepared in recombinant form by expression in a host cell. Suitable expression methods are well known to those of skill in the art and many are described in detail by Sambrook et al (2000) and Fernandez & Hoeffler (1998).

The proteins or functional equivalents of the present invention can also be prepared using conventional techniques of protein chemistry, for example by chemical synthesis.

According to a second aspect of the invention, there is provided a nucleic acid molecule comprising a nucleotide sequence encoding an ICMM or functional equivalent thereof, according to the first aspect of the invention. Such nucleic acid molecules include single- or double-stranded DNA, cDNA and RNA, as well as synthetic nucleic acid species. Preferably, the nucleic acid molecules are DNA or cDNA molecules.

The invention also includes cloning and expression vectors incorporating the nucleic acid molecules of the second aspect of the invention. Such expression vectors may additionally incorporate the appropriate transcriptional and translational control sequences, for example enhancer elements, promoter-operator regions, termination stop sequences, mRNA stability sequences, start and stop codons or ribosomal binding sites, linked in frame with the nucleic acid molecules of the second aspect of the invention.

Additionally, it may be convenient to cause a recombinant protein to be secreted from certain hosts. Accordingly, further components of such vectors may include nucleic acid sequences encoding secretion, signalling and/or processing sequences.

Vectors according to the invention include plasmids and viruses (including both bacteriophage and eukaryotic viruses), as well as other linear or circular DNA carriers, such as those employing transposable elements or homologous recombination technology. Many such vectors and expression systems are known and documented in the art (see, for example, Fernandez & Hoeffler, 1998). Particularly suitable viral vectors include baculovirus-, adenovirus- and vaccinia virus-based vectors.

Suitable hosts for recombinant expression include commonly used prokaryotic species, such as E. coli, or eukaryotic yeasts that can be made to express high levels of recombinant proteins and that can easily be grown in large quantities. Mammalian cell lines grown in vitro are also suitable, particularly when using virus-derived expression systems. Another suitable expression system is the baculovirus expression system, that involves the use of insect cells as hosts. An expression system may also constitute host cells that have the appropriate encoding nucleic acid molecules incorporated into their genome. Proteins, or protein fragments may also be expressed in vivo, for example in insect larvae or in mammalian tissues.

A variety of techniques may be used to introduce the vectors according to the present invention into prokaryotic or eukaryotic host cells. Suitable transformation or transfection techniques are well described in the literature (see, for example, Sambrook et al, 2000; Ausubel et al, 1991; Spector, Goldman & Leinwald, 1998). In eukaryotic cells, expression systems may either be transient (e.g. episomal) or permanent (such as by chromosomal integration) according to the needs of the system.

The invention also includes transformed or transfected prokaryotic or eukaryotic host cells containing a nucleic acid molecule as defined above.

A further aspect of the invention provides a method for preparing an ICMM or a functional equivalent thereof as defined above, which comprises culturing a host cell containing a nucleic acid according to the invention under conditions whereby said protein is expressed and recovering said protein thus produced.

A further aspect of the invention provides a method for isolating an ICMM or functional equivalent thereof, as defined above comprising the steps of: preparing an extract from a haematophagous arthropod as defined previously; separating said extract into fractions containing different proteins; testing said fractions for the ability to modulate, preferably inhibit, ion channel activity; and isolating said ICMM or functional equivalent thereof, from a fraction(s) that possesses the ability to modulate, preferably inhibit, ion channel activity.

Preferably, said extract is a salivary gland extract. Depending on the arthropod species the preparation of such salivary gland extracts may take place at any one of several suitable points in the feeding cycle. In the case of Hybomitra bimaculata, the extract is preferably prepared from the salivary glands of adult females since this is the only haematophagous instar.

In a particularly preferred embodiment of the invention, an ICMM or functional equivalent thereof may be obtained by a method comprising the steps of:
a) preparing a salivary gland extract from a haematophagous arthropod;
b) separating said extract into fractions containing proteins;
c) testing said fractions for the ability to modulate the activity of an ion channel; and
d) isolating said ICMM or functional equivalent thereof from a fraction(s) that possesses the ability to modulate ion channel activity.

Preferably, the haematophagous arthropod used in the above method is a horsefly of the Tabinadae family. Preferably, the ICMMs or functional equivalents, including EV048 and homologues thereof, are derived from horseflies of the Hybomitra, Heptatoma, Chrysops, Haematopota and Tabanus genera using the above-described method, more preferably, from Hybomitra bimaculata.

Suitable methods of separating haematophagous arthropod extracts into fractions containing purified proteins will be apparent to those skilled in the art. Preferably, the extract is separated into fractions of may be carried out using a chromatographic procedure, such as fast phase liquid chromatography (FPLC), high-performance liquid chromatography (HPLC), ion exchange chromatography, affinity chromatography, gel filtration or reverse phase HPLC.

Testing of the fractions for their ability to modulate ion channel activity, in particular to cause vasodilation and/or positive inotropism and/or lengthen action potential, can also be carried out by one of several methods known to those skilled in the art. The methods used for assessing whether modulation of ion channels has been effected may vary depending on the ion channel under consideration. For example, in the case of the sodium-potassium ATPase, the activity of ATPase on varying concentrations of ATP in the presence and absence of fractions containing putative ICMMs may be assessed. In the case of inotropism the effect of fractions on whole cell patch clamping in isolated cardiomyocytes or the effect on left ventricular output in an isolated perfused Langendorf rat heart may be assessed. The effect on lengthening of action potential may be assessed by whole cell patch clamping in isolated cardiomyocytes. Vasodilation may be assessed by the effect of the fractions containing putative ICMMs on pre-contracted rat femoral artery rings or by assessing the effect of fractions on coronary blood flow in an isolated Langendorf heart.

Following identification of a fraction that possesses the ability to modulate ion channel activity, an ICMM or functional equivalent thereof may be isolated by any suitable procedures, including procedures such as SDS-polyacrylamide gel electrophoresis or two dimensional gel electrophoresis.

The present invention also includes an ICMM or functional equivalent thereof obtainable by any one of the methods described above. Preferably, the ICMM or functional equivalent thereof modulates the activity of a sodium channel, a potassium channel, a calcium channel and/or a sodium-potassium ATPase. The ICMM or functional equivalent thereof may modulate the activity of more than one ion channel. The ICMM or functional equivalent thereof exhibits the preferred ion channel modulatory properties listed above.

The method set out above may further comprise isolating and sequencing a gene encoding an ICMM, or functional equivalent thereof obtained using any of the methods set out above. For example, an isolated and purified ICMM may be subjected to a step of amino acid sequencing, followed by screening of a salivary gland gene library, for example using the polymerase chain reaction to isolate a gene encoding the ICMM. One example of a suitable procedure is by screening a cDNA library, optionally a cDNA expression library. Certain expression libraries can be designed to generate tagged arthropod proteins, so facilitating their analysis and purification. Suitable procedures for the preparation and isolation of parasite proteins can be found, for example, in co-owned patent applications PCT/GB97/01372 and PCT/GB98/03397. A variety of suitable procedures for isolating a gene encoding an ICMM according to the invention will be known to the skilled reader.

In a particularly preferred embodiment of the invention, a gene encoding an ICMM or functional equivalent thereof may be obtained by a method comprising performing the steps outlined in detail above to isolate the ICMM or functional equivalent thereof, and additionally performing the steps of:
e) obtaining the N-terminal sequence of said isolated ICMM or functional equivalent thereof;
f) designing a degenerate oligonucleotide; and
g) using said degenerate oligonucleotide to screen a salivary gland gene library to isolate a gene encoding the ICMM or functional equivalent thereof.

Once the ICMM or functional equivalent thereof has been isolated, sequencing of the N-terminus of the protein may be carried out by any suitable method, as will be apparent to those skilled in the art. Following the determination of the N-terminal sequence of the ICMM, or functional equivalent thereof, a skilled person will readily be able to design one or more degenerate oligonucleotide probes or degenerate PCR primers which could encode this peptide sequence. These primers may then be used to screen a salivary gland gene library, for example by hybridisation or by PCR. Preferably, such a library is a cDNA gene library.

According to a further aspect of the invention there is provided a composition comprising an ICMM or functional equivalent, or a nucleic acid comprising a nucleotide sequence encoding an ICMM or a functional equivalent according to the invention in conjunction with a pharmaceutically acceptable carrier.

Pharmaceutically-acceptable carriers include any carrier that does not itself induce the production of antibodies harmful to the individual receiving the composition. Suitable carriers are typically large, slowly metabolised molecules such as proteins, polysaccharides, polylactic acids, polyglycolic acids, polymeric amino acids, amino acid copolymers, lipid aggregates (such as oil droplets or liposomes) and inactive virus particles. Such carriers are well known to those of skill in the art.

In certain circumstances, such a composition may be used as a vaccine, and may thus optionally comprise an immunostimulating agent, for instance an adjuvant of the type referred to below. According to a further aspect of the invention, there is provided a process for the formulation of a vaccine composition comprising bringing an ICMM or functional equivalent, or a nucleic acid comprising a nucleotide sequence encoding an ICMM or a functional equivalent according to the invention into association with a pharmaceutically-acceptable carrier, optionally with an adjuvant. Suitable adjuvants are well-known in the art and include oil-in-water emulsion formulations, saponin adjuvants, Complete Freund's Adjuvant (CFA), Incomplete Freund's Adjuvant (IFA) and other substances that may act as immunostimulating agents to enhance the effectiveness of the vaccine composition.

According to a further aspect, the present invention provides for the use of an ICMM or functional equivalent thereof in therapy.

The invention also provides a method of treating an animal suffering from a disease or condition caused by a fault in ion channel activity, comprising administering to said animal an ICMM or functional equivalent thereof, a nucleic acid comprising a nucleotide sequence encoding an ICMM or a functional equivalent thereof or a pharmaceutical composition according to the invention in a therapeutically effective amount. Preferably, said animal is a mammal, more preferably a human.

Examples of conditions suitable for treatment using the ICMMs or functional equivalents of the invention include cardiac conditions such as coronary insufficiency leading to angina, congestive cardiac failure and cardiac arrhythmias; peripheral vascular disease such as cerebro-vascular insufficiency, intermittent claudication and Buerger's disease; vasospastic disorders such as Raynaud's disease, cerebral or coronary vasospasm; reperfusion following stroke and myocardial infarction; shock including septic shock, haemorrhagic shock and cardiogenic shock; hypertension; to assist in circulatory support during and following cardio-pulmonary by-pass or angioplasty procedures.

The invention also includes the use of an ICMM, variant or functional equivalent thereof as a diagnostic tool. In particular, the invention includes the use of an ICMM, variant or functional equivalent thereof in the diagnosis of abnormalities of the cardiovascular system. Methods of diagnosis using an ICMM, variant or functional equivalent thereof will be well known to those skilled in the art.

The present invention also includes the use of an ICMM, variant or functional equivalent thereof, as a tool in the study of ion channel modulation and the effects of ion channel modulation, including vasodilation and inotropism.

Various aspects and embodiments of the present invention will now be described in more detail by way of example, with particular reference to ICMMs isolated from horseflies and especially from H. bimaculata. It will be appreciated that modification of detail may be made without departing from the scope of the invention.

### BRIEF DESCRIPTION OF THE DRAWINGS

FIG. 1: Results of HPLC fractionation of crude Hybomitra bimaculata salivary gland extract. In the first purification, SGE was applied to a Vydac C-4, 250×4.6 mm ID, 5 μm particle size column, UV monitored at 210 nm and 220 nm. A gradient of 10-100% ACN with 0.1% TFA, flow rate 1 ml/min and with 1% ACN/min increments was used (FIG. 1A). The active fraction from the first purification was further purified using a Beckman Ultrasphere C-18, 250×4.6 mm ID, 5 μm particle size column and a gradient of 10-40% ACN with 0.1% TFA, flow rate 1 ml/min with 0.5% ACN/min increments, and monitored at 210 and 220 nm (FIG. 1B). For the third purification, a Vydac C 18, 250×4.6 mm ID, 5 μm particle size column was used under the same conditions as for the second purification (FIG. 1C).

FIG. 2: In vitro effect of salivary gland extract (SGE) from Hybomitra bimaculata on the cardiac Na,K-ATPase activity. The SGE-quantity is expressed as amount of applied proteins from the extract.

FIG. 3: In vitro effect of salivary gland extract (SGE) of Hybomitra bimaculata on kinetic parameters of Na,K-ATPase from rat heart. The SGE-quantity is expressed as amount of applied proteins from the extract. The data represent means±SEM of 3 estimations, p<0.05.

FIG. 4: Vasodilating activity of Hybomitra bimaculata salivary gland extract on isolated rat femoral arterial rings with and without endothelium.

FIG. 5: Vasodilating activity of protein HPLC fractions (retention time 10-28 minutes) of Hybomitra bimaculata salivary gland extracts on rat arterial rings without endothelium.

FIG. 6: Effect of 100 μL Hybomitra bimaculata salivary gland extract on coronary blood flow in the isolated perfused rat heart.

FIG. 7: Reverse phase HPLC of crude salivary gland extract of Hybomitra bimaculata. The peaks containing vasodilator activity are indicated with retention times.

FIG. 8: N-terminal amino acid sequence of Hybomitra bimaculata salivary gland product EV048. Tentatively assigned residues after position 41 are indicated with a question mark.

FIG. 9: Primary structure of Hybomitra bimaculata peptide EV048 (FIG. 9a). Signal sequence underlined, cysteine residues shown in bold type, stop codon indicated by an asterisk. Alignment of peptide EV048 with Pfam consensus sequence for Kazal type proteins (FIG. 9b).

FIG. 10: Pfam alignment of Hybomitra bimaculata peptide Ev049 with Kazal type proteins. Gaps in the alignment are indicated by dashes and dots. Residues in lower case are outstandingly different from the overall consensus. THBI_RHOPR/6-48 and 57-101, Rhodnius prolixus thrombin inhibitor domain 1+2; IELA_ANESU/4-48, Anemonia sulcata inhibitor of elastase; AGRI, agrin; IAC, acrosin inhibitor; IOVO, ovomucoid inhibitor; QR1, quail retinal 1; SC1, secreted calcium binding 1 matric glycoprotein; SPRC, secreted protein acdic and rich in cysteine also called osteonedinand basement membrane protein 40 (bm40).

FIG. 11: Coomassie blue stained NuPAGE 4-12% Bis-Tris gel showing purified peptide EV048. Lane (1) marker, (2-5) fractions h-k which elute at about 0.15M NaCl from a SP sepharose column. Marker sizes (kDa) indicated on left.

FIG. 12: Effect of EV048 on isolated rat cardiomyocytes. Results of three experiments.

FIG. 13: Effect of EV048 on coronary blood flow in isolated perfused rat heart.

FIGS. 13a and 13b show the results of two separate experiments.

### EXAMPLES

Materials and Methods

Horse Fly Collection

Horse flies were collected during the summer of 1999 in selected sites of south-western and western Slovakia using Manitoba traps. The effectiveness of the traps was improved by application of CO₂. Collections were performed during optimal weather conditions (sunny days, temperature 24-28° C., no wind) from May until the end of August. The collecting day started at 9:00 a.m. and finished at 5:00 p.m. Approximately 100-150 female horse flies per trap were collected each day of trapping, resulting in a total of 5,394 specimens. Horse flies were transported to the laboratory alive and then immediately processed to identify and isolate those of the species Hybomitra bimaculata.

Salivary Gland Sample Preparation and Purification

Prior to the dissection of salivary glands, horse-flies were immobilized for a few minutes by placing them at 4° C. The salivary glands were dissected under a microscope and transferred to Eppendorf vials with cooled PBS buffer (0.01M phosphate buffer and 0.15M NaCl, pH 7.2). Samples were heated in a 80° C. water bath for 5 min, homogenized and centrifuged at 2,500 g for 10 min. The supernatant (referred to as crude salivary gland extract, SGE) was collected and stored at −70° C. or immediately filtered through a Millex-LG syringe driven filter unit (0.20 μm, 4 mm) and processed by HPLC.

Reverse Phase (RP-) HPLC

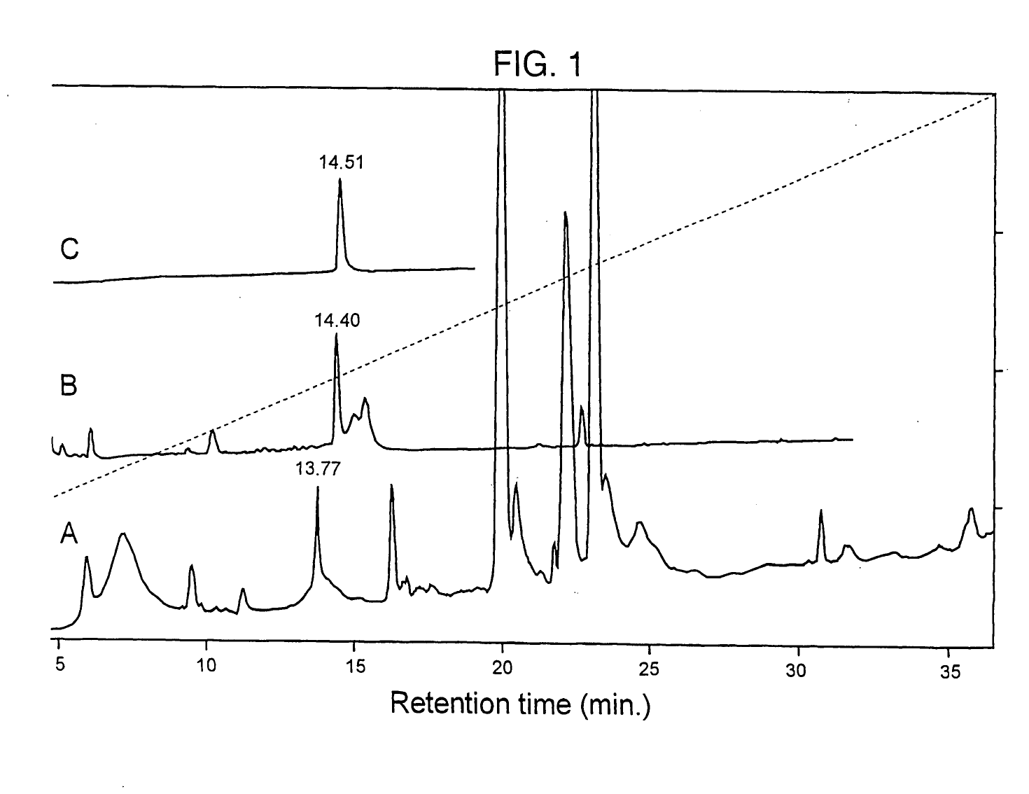

For purification and identification of vasoactive compounds, SGE of Hybomitra bimaculata horse flies was used. The SGE samples were diluted in 500 μl of 10% acetonitrile (ACN) with 0.1% trifluoroacetic acid (TFA) (buffer A) and loaded onto a Beckman Instruments 126/168 DAD HPLC system. In the first purification, SGE was applied to a Vydac C-4, 250×4.6 mm ID, 5 μm particle size column, UV monitored at 210 nm and 220 nm. A gradient of 10-100% ACN with 0.1% TFA, flow rate 1 ml/min and with 1% ACN/min increments was used (FIG. 1A). The active fraction from the first purification was further purified using a Beckman Ultrasphere C-18, 250×4.6 mm ID, 5 μm particle size column and a gradient of 10-40% ACN with 0.1% TFA, flow rate 1 ml/min with 0.5% ACN/min increments, and monitored at 210 and 220 nm (FIG. 1B). For the third purification, a Vydac C 18, 250×4.6 mm ID, 5 μm particle size column was used under the same conditions as for the second purification (FIG. 1C). Fractions were collected and dried in a Savant Instruments Speed-Vac.

Protein Sequence Analysis

Protein sequence analysis was performed by N-terminal Edman degradation using an automated sequencer (Model 494 Applied Biosystems) by Eurosequence (Groningen, the Netherlands). Reagents, chemicals and materials were obtained from Applied Biosystems (Warrington, U.K and Foster City, Calif., U.S.A).

Construction of H. bimaculata cDNA Library

One hundred pairs of H. bimaculata salivary glands were excised as described above and placed in 1 ml RNAlater® (Ambion) (in place of PBS) and stored at −20° C. mRNA was isolated using the FastTrack™ 2.0 mRNA isolation kit (Invitrogen) and cDNA was synthesised using a Stratagene cDNA synthesis kit (Cat # 200401-5). After fractionation into large and small cDNAs on a Sepharose CL-2B column, the ethanol precipitated cDNA pellets were each resuspended in 3.5 μl ddH₂O. cDNA yields were approximately 30 ng/μl and 200 ng/μl for the large and small molecules, respectively. Two μl of the large and 0.5 μl of the small cDNA were ligated into the Stratagene UniZAP XR phage vector (Cat. # 237211) and packaged with Gigapack® III Gold packaging extract. There were 31100 primary plaques in the large cDNA library and 524,000 primary plaques in the small cDNA library. After amplification the titres of the large and small libraries were 8.7×10⁹ pfu/ml and 9.7×10⁹ pfu/ml respectively.

Twenty plaques from each library were picked into 0.5 ml SM buffer (0.1M NaCl, 8 mM MgSO₄, 50 mM TRIS.HCl pH 7.5, 0.01% gelatin) 1% chloroform and eluted from agarose plugs by vortexing. Phage insert sizes were examined by PCR using T7 primers (5′TAA TAC GAC TCA CTA TAG 3′) and T3 (5′AAT TAA CCC TCA CTA AAG 3′). Each 100 μl reaction comprised 2 μl eluted phage, 2 μl 10 mM dNTPs, 2 μl of each primer (from stocks of 0.5 μg/ml), 10 μl 10×REDTaq (Sigma) PCR reaction buffer (100 mM Tris-HCl pH 8.3, 500 mM KCl, 11 mM MgCl₂, 0.1% gelatin), 3 μl REDTaq (Sigma) DNA polymerase (1 unit/μl in 20 mM Tris-HCl, pH 8.0, 100 mM KCl, 0.1 mM EDTA, 1 mM DTT, 0.5% Tween 20, 0.5% Igepal CA-630, inert dye, 50% glycerol) and 79 μl ddH₂O. Thermal cycling (Hybaid Touchdown thermal cycler) parameters were 1×94° C. 4 min, 30×94° C. 1 min, 48.5° C. 45 s, 72° C. 90 s, and 1×72° C. 5 min. Agarose gel electrophoresis of the PCR products showed that large library inserts were ≧1600 base pairs and small library inserts ≦1600 base pairs.

Cloning cDNA of EV048

The N-terminal sequence determined for the HPLC fraction collected at 14.51 min (designated EV048) from H. bimaculata SGE was used to design three degenerate primers (HF1, HF2, HF3) for use with the T7 primer (which binds to the UniZAP XR vector), to amplify the cDNA encoding the peptide. The sequences of the primers were HF1 5′GAY GAR TGY CCN MGN ATN TG 3′, HF2 5′GAR TGY CCN MGN ATN TGY AC 3′, and HF3 5′ACN TTY GGN AAY CAR TG 3′ (where Y═C or T, R=G or A, N=A or C or G or T, and M=A or C). Each 10011 reaction comprised 3 μl large or small library, 3 μl 10 mM dNTPs, 2 μl T7 and 4 μl HF1 or HF2 or HF3 (from stocks of 0.5 μg/ml), 10 μl 10×REDTaq PCR reaction buffer, 3 μl REDTaq DNA polymerase and 75 μl dH₂O. Thermal cycling parameters were 1×94° C. 4 min, 30×94° C. 1 min, 48.5° C. 45 s, 72° C. 90 s, and 1×72° C. 5 min.

Agarose gel electrophoresis revealed a range of PCR products. Five of these products were purified using a Qiaex II gel extraction kit (Qiagen) and sequenced with an ABI PRISM™ dye terminator cycle sequencing ready reaction kit and ABI sequencer (Perkin Elmer). Conceptual translation of one of the PCR products, derived from the small cDNA library using primer HF2 with T7, revealed an exact match with the N-terminal sequence of EV048. The sequence extended beyond the stop codon of the cDNA encoding the peptide. A reverse primer (HR1 5′AAT ACA ACA TAT TCA AGT GG 3′) matching the region beyond the stop codon was used with the T3 primer (which binds to the UniZAP XR vector) to obtain the 5′ end of the cDNA. The PCR product was cloned into the pGEM®-T Easy vector (Promega) then sequenced revealing a full-length cDNA encoding EV048.

Sequence Analysis

Analyses were carried out using the GCG suite of programs (Wisconsin Package Version 10.1, Genetics Computer Group (GCG), Madison, Wis.) and also the ExPASy (Expert Protein Analysis System) proteomics server of the Swiss Institute of Bioinformatics (http://expasy.hcuge.ch/).

BAC-BAC® Baculovirus Expression and Purification of EV048

The HR1/T3 PCR product cloned into pGEM®-T Easy was amplified using primer HF6 (5′ GTA CGG ATC CAT GAA ATT TGC CTT GTT CAG T 3′) which matches the signal sequence of EV048 and has a Bam HI restriction enzyme site (italic), and primer HR3 (5′ CAT GCT GCA GTT AGT GAT GGT GAT GGT GAT GAC CCT TGC ACT CGC CAT CATG 3′) which matches the sequence encoding the carboxy-terminal end of the protein and includes a codon for a glycine followed by six histidine residues (underlined) then a stop codon (bold) and PstI restriction enzyme site (italic). The 100 μl reaction comprised 1 ng HR1/T3 PCR product in pGEM®-T Easy in a volume of 1 μl, 2.5 μl 10 mM dNTPs, 2 μl HF6 and 2 μl HR1 or HF2 (from stocks of 0.5 μg/ml), 10 μl 10×REDTaq PCR reaction buffer, 3 μl REDTaq DNA polymerase and 77.5 μl dH₂O. Thermal cycling parameters were 1×94° C. 4 min, 20×94° C. 1 min, 48.5° C. 45 s, 72° C. 90 s, and 1×72° C. 5 min. The PCR product was gel purified, digested with Bam HI and Pst I and inserted into Bam HI and Pst I cut pFastBac1 plasmid (Gibco-BRL®). The sequence of the construct was verified by sequencing with primers PFBR (5′ GAT TAT GAT CCT CTA GTA C₃′) and PFBF (5′ TAT TCC GGA TTA TTC ATA CC 3′) which match pFastBac1 either side of the multiple cloning site of the plasmid. Transformation of the DH10α bacteria carrying the baculovirus-DNA, purification of the baculovirus DNA and generation of high titre baculovirus stock were performed in accordance with the instructions accompanying the BAC-BAC® baculovirus expression system (Gibco-BRL®).

For expression, Sf9 cells grown in Sf-900 II serum free medium (Gibco-BRL®) to a cell density of 1×10⁶ cells/ml were infected at a multiplicity of infection of 5 and grown for a further 60 h. For purification, the cultures were centrifuged in a JA-20 rotor at 3000 RPM for 10 min and the supernatant was poured into beakers kept on ice. Polyethylene glycol MW 3350 was added to 30% (w/v) with stirring and the mixture was stirred for one hour. The mixture was centrifuged in a JA-20 rotor at 5000 RPM for 20 min and the protein pellet was resuspended in 20 ml binding buffer (50 mM Na₂HPO₄/NaH₂PO₄ pH 8, 500 mM NaCl, 10% glycerol) per gram of wet paste. After addition of 50011 per gram of wet paste TALON Metal Affinity Resin (Clontech) the resuspended pellet was rocked for 1 hour on ice. The resin-binding buffer mix was brought onto a disposable 1 ml purification column (Invitrogen). The resin was then washed with 10 volumes 10 mM TRIS.HCl pH 8 and then with 15 volumes of wash buffer (50 mM Na₂HPO₄/NaH₂PO₄ pH 6.5, 500 mM NaCl, 10% glycerol). Bound proteins were eluted with 6 volumes 100 mM NaH₂PO₄, 250 mM imidazole and concentrated using Centricon 3 centrifugal filter devices (Amicon) spun in a JA-12 rotor at 5000 RPM for 4 hours. Peptide EV048 was then purified further by cation exchange chromatography. The buffer flow rate was 1 ml/min for the washing and loading steps. An SP Sepharose cation exchange column (Pharmacia) was washed with 10 column volumes of running buffer (50 mM Na₂HPO₄/NaH₂PO₄ pH 6.8) and the concentrated protein was diluted 200-fold in running buffer then passed over the column. After washing with a further 10 column volumes of running buffer, proteins were eluted using a 30 min 0-0.75M NaCl gradient at a flow rate of 0.5 ml/min. Peptide EV048 eluted as a single peak which was concentrated and visualised on a 4-12% Bis-Tris polyacrlamide gel (Novex-Invitrogen) stained with GelCode Blue (Sigma).

Effect of Crude Hybomitra bimaculata Salivary Gland Extract (SGE) on Sarcolemmal Na, K-ATPase

Cardiac sarcolemma was prepared from samples of the hearts of Wistar Kyoto rats by the hypotonic shock-NaI treatment method previously described (Vrbjar et al 1984). The protein content was assayed by the procedure of Lowry (Lowry et al 1951) using bovine serum albumin as a standard.

The substrate kinetics of Na,K-ATPase were estimated by measuring the splitting of ATP by 30-50 μg sarcolemmal proteins at 37° C. in the presence of increasing concentrations of ATP in the range 0.08-4.0 mmol/l in a total volume of 0.5 ml of medium containing 50 mmol/l imidazole (pH 7.4), 4 mmol/l MgCl₂, 10 mmol/l KCl and 100 mmol/l NaCl. After 15 minutes of preincubation in the substrate free medium, the reaction was started by addition of ATP and 15 minutes later it was terminated by the addition of 1 ml of 12% solution of trichloroacetic acid. Verification of the time dependence of ATP-hydrolysis showed that for up to 20 minutes the ATP splitting was linear in the whole ATP concentration range applied. The inorganic phosphorous liberated was determined according to Taussky and Shorr (1953). In order to establish the Na,K-ATPase activity, the ATP hydrolysis that occurred in the presence of Mg²⁺ only was subtracted. From each sarcolemmal preparation three individual Kₘ and Vₘₐₓ values were obtained.

Crude salivary gland extracts (SGE) was obtained from Hybomitra bimaculata. The influence of this extract on the function of Na,K-ATPase was tested in vitro by addition of 0-10 μg of SGE to 30 μg of sarcolemmal proteins.

The kinetic parameters were evaluated by direct nonlinear regression of the data obtained. All results were expressed as mean ±SEM. The significance of differences between individual groups was determined by one way Bonferroni test (Sahai & Ageel, 2000), a value of p<0.05 being regarded as significant.

Isolated Heart Preparation and Perfusion Technique

Rat hearts were rapidly excised, placed in ice-cold perfusion buffer, cannulated via the aorta and perfused in the Langendorff mode at a constant perfusion pressure of 70 mm Hg and at 37° C. Perfusion solution was a Krebs Henseleit buffer gassed with 95% O₂ and 5% CO₂ (pH 7.4) containing (in mM): NaCl 118.0, KCl 4.7, MgSO₄ 1.66, CaCl₂ 2.52, NaHCO₃ 24.88, KH₂PO₄ 1.18 and glucose 5.55. The solution was filtered through a 5 μm porosity filter (millipore).

An epicardial electrogram (EG) was registered by means of two stainless steel electrodes attached to the apex of the heart and the aortic cannula and continuously recorded (Miograph ELEMA-Siemens, Solna, Sweden). Heart rate was calculated from the EG.

Coronary flow was measured by a timed (10 s interval) collection of coronary effluent which was weighed on an electronic balance (AND HF 200 G, A&D Company Limited). Left ventricular pressure was measured by means of a latex water-filled balloon inserted into the left ventricle via the left atrium (adjusted to obtain end-diastolic pressure of 5-10 mm Hg) and connected to a pressure transducer (P23 Db Pressure Transducer, Gould Statham Instruments, Inc.)

Crude SGE of Hybomitra bimaculata was investigated. SGE from 8-20 salivary glands made up to 200 μl was injected through a syringe directly into the aortic cannula with continuous measurement of coronary flow (CF), left ventricular pressure (LVP) and EG. Changes in CF, LVP and heart rate (HR) were evaluated.

Isolated Rat Femoral Artery Preparation

Wistar rats 12 weeks old of both sexes were used. After they were killed humanely, two segments (each approx. 10 mm in length) of femoral artery were isolated and placed in a Krebs-Ringer bicarbonate solution comprising 118 mM NaCl, 5 mM KCl, 25 mM NaHCO₃, 1.2 mM MgSO₄.7H₂O, 1.2 mM KH₂PO₄, 2.5 mM CaCl₂, 1 mM EDTA, 1.1 mM ascorbic acid, and 11 mM glucose. The endothelium was removed from one half of each segment preparation by gently rubbing the intimal surface. The vessel segments were cleaned of adherent connective tissue and cut into 3 mm ring segments. Two stainless-steel wires were passed through the lumen taking care not to damage the endothelium. The rings were mounted on a myograph capable of measuring the isometric wall tension and placed within a bath containing Krebs-Ringer solution through which was bubbled 95% O₂ and 5% CO₂, maintained at a temperature of 37° C. and pH 7.4. The effectiveness of endothelium removal was demonstrated by failure of acetylcholine (5×10⁻⁶ mol/l) to relax a contraction induced by phenylephrine (5×10⁻⁶ mol/l). The plateau of the contractile response induced by phenylephrine (5×10⁻⁶ mol/l) was taken as a measure of 100% contraction.

Effects of SGE on Isolated Rat Ventricular Myocytes

Rat ventricular myocytes were isolated by enzymatic digestion, layered in a glass perfusion chamber mounted on the stage of an inverted microscope, and perfused with a salt solution containing (mM): NaCl 140, KCl 54, CaCl₂ 1.0, MgCl₂ 1.0, glucose 10.0 and HEPES 10.0 (pH 7.35 adjusted with NaOH). The cells were field stimulated to contract at 0.5 Hz with 1.0 ms square wave pulses of supra-threshold voltage. The decrease in cell length during each contraction was measured with an edge detection system using a photodiode array. The temperature of the superfusate was 24±2° C. Data were digitised and acquired on a personal computer via an A/D converter using VCLAMP software (CED, Cambridge, UK).

To measure action potential and membrane currents, an Axoclamp 200 amplifier (Axon Instruments, Inc., Burlingame, Calif., USA) was used in whole-cell voltage clamp or current clamp mode to record ionic currents and action potentials. Patch pipettes were pulled from filamented borosilicate capillary glass (GC150TF; Clark Electromedical Instruments, UK) on a microprocessor-based three-stage puller (Mecanex BB-CH-PC, Basel, Switzerland). The pipettes had resistances of 2-4 MΩ after filling with internal solution.

For action potential recordings, the pipettes were filled with a solution containing (in mM): KCl 140, MgCl₂ 1.0, Mg-ATP 5.0, Na₂-phosphocreatine 5.0, HEPES 5.0 (pH 7.2 adjusted with KOH). The external solution for these experiments was (mM): NaCl 140, KCl 5.4, CaCl₂ 1.8, MgCl₂ 1.0, glucose 10.0 and HEPES 5.0 (pH 7.4 adjusted with NaOH). Action potentials were elicited by injection of short current pulses.

To isolate whole-cell I_{Ca} the pipette was filled with a solution containing (mM): CsCl 120, MgCl₂ 1.0, Mg-ATP 5.0, Na₂-phosphocreatine 5.0, EGTA 10.0 and HEPES 5.0 (pH 7.2 adjusted with CsOH). Cells were superperfused with a solution containing (mM): tetraethylammonium-Cl 140, CaCl₂ 1.8, MgCl₂ 1.0, glucose 10.0, HEPES 5.0, and 4-aminopyridine 3.0 (at pH 7.4 adjusted with tetraethylammonium hydroxide). I_{Ca} was elicited by applying 100 ms depolarising voltage pulses in 5 mV steps from a holding potential of −45 mV. To measure whole-cell Current_{transient outward} (Iₜₒ), the pipette was filled with a solution containing (mM): KCl 145, MgCl₂ 3.0, Mg-ATP 5.0, Na₂-phosphocreatine 5.0, EGTA 5.0 and HEPES 5.0 (pH 7.2 adjusted with KOH). Cells were superperfused with a solution containing (mM): choline-Cl 145, KCl 5.4, CaCl₂ 0.5, MgCl₂ 0.5, glucose 5.5, CoCl₂ 2.0 and HEPES 5.0 (pH 7.4 adjusted with KOH). Iₜₒ was elicited by applying 100 ms depolarising voltage pulses in 10 mV steps to +70 mV from a holding potential of −80 mV. In these experiments, the interval between pulses was 5 seconds and the superfusate temperature was 35±1° C.

The contractile and electrophysiological effects of the 56 amino acid recombinant peptide from Hybomitra bimaculata (EV048) was tested on single cardiomyocytes isolated from rat ventricles.

Results

Horse Fly Collection

In Slovakia, 63 horse fly species have been recorded. Approximately one third of these species are very common, one third are common in appropriate biotopes, and the remaining species are rare. In this collection, 16 horse fly species were present of which Hybomitra bimaculata was the second most abundant species, a total of 1300 individuals being collected.

Effect on Sarcolemmal Na,K-ATPase

The influence of salivary gland extract (SGE) from Hybomitra bimaculata on the function of Na,K-ATPase was tested by the addition of various amounts of SGE to 30 μg of sarcolemmal proteins. Na,K-ATPase, an enzyme involved in the active translocation of Na⁺ and K⁺ ions across cell membranes causes the potassium dependent relaxation or so-called hyperpolarisation. For this purpose the enzyme utilises the energy derived from hydrolysis of ATP. Therefore, in the present study attention was focused on the influence of SGE on the ATP-binding properties of the enzyme by investigating its behaviour in the presence of increasing concentrations of ATP.

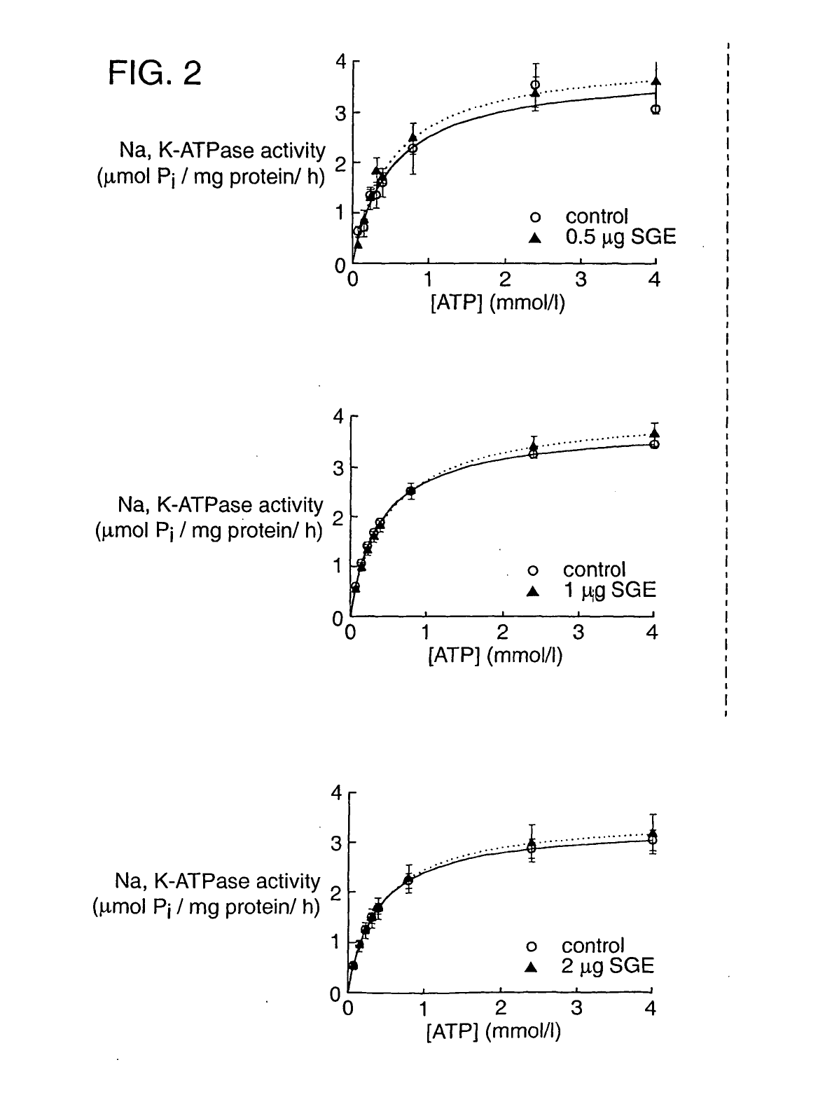

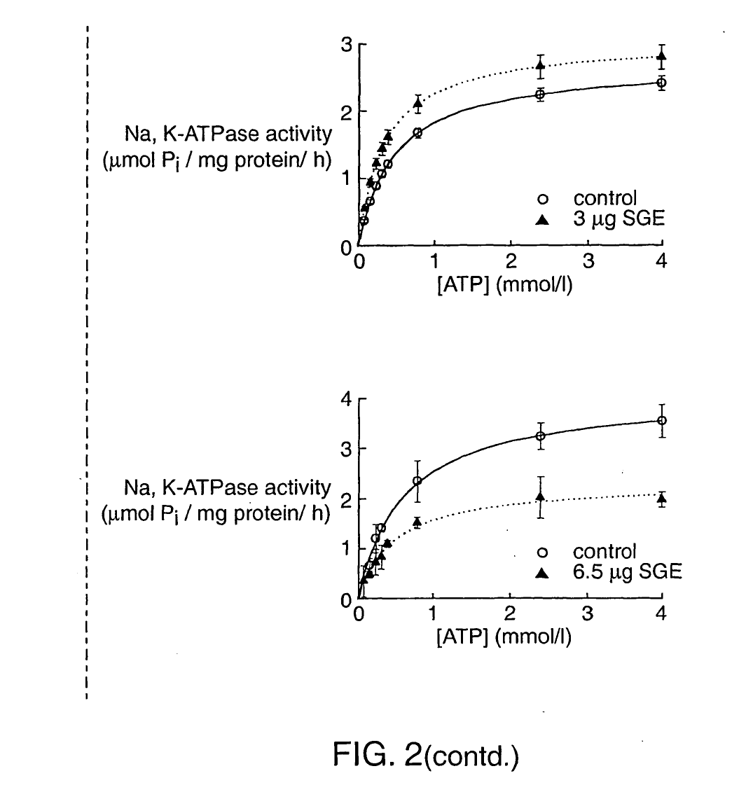

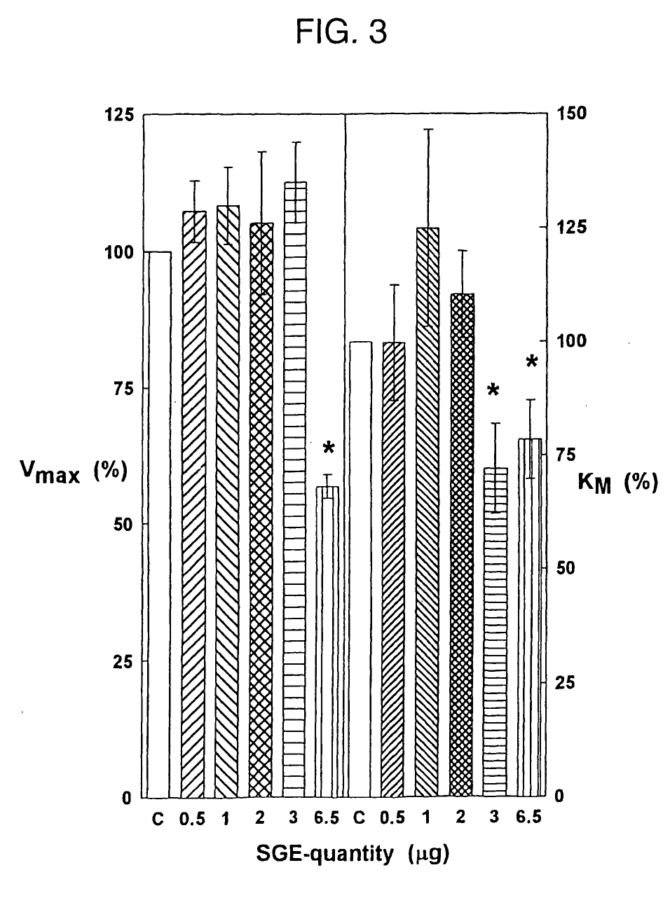

The result of this experiment is shown in FIGS. 2 & 3. The result clearly showed that SGE from Hybomitra bimaculata contains at least one compound which at lower concentrations stimulates Na,K-ATPase but at the highest concentration tested (6.5 μg) has an inhibitory effect. This biphasic reaction suggests that at lower concentrations the salivary gland extract is able to increase the hyperpolarisation of muscle cells but at higher concentrations it has the reverse effect.

Vasodilating Activity of Rat Femoral Artery Induced by SGE

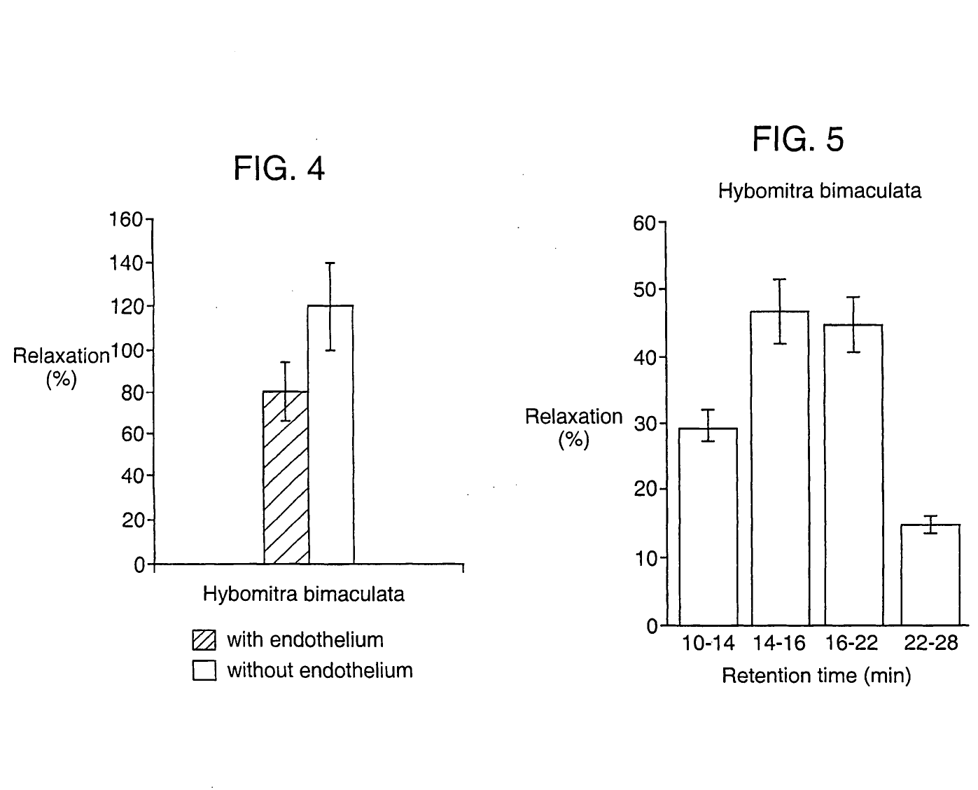

The relaxing responses of rat femoral artery induced by SGE from Hybomitra bimaculata was examined. Using rat femoral artery with intact endothelium, the application of 50 μl SGE (equivalent to ½ salivary gland) from Hybomitra bimaculata induced 119% relaxation (FIG. 4).

Removal of endothelium did not decrease the vasodilating responses induced by SGE. In fact, SGE from Hybomitra bimaculata induced 39% (p<0.05) greater relaxation of the artery than with the endothelium intact (FIG. 4). In view of this finding the effects of fractions obtained by HPLC on endothelium-denuded rings was investigated.

The vasodilating responses of endothelium-denuded arterial rings induced by protein HPLC fractions of salivary glands from Hybomitra bimaculata obtained in the retention time range 10-28 min were compared. The maximum vasodilating responses were induced by a fraction with the retention time 13.77 min (47% relaxation; FIG. 5).

Effect of Hybomitra bimaculata SGE on NaHPO₄ Perfused Isolated Rat Heart

The underlying principle of the Langendorff model of perfusion of isolated rat heart (Langendorff, 1895) is to force blood, or any other oxygenated fluid appropriate to maintain cardiac activity, towards the heart through a cannula inserted into the ascending aorta. Retrograde perfusion closes the aortic valves (mimicking the in situ heart during diastole) and the perfusate is displaced through the coronary arteries. After passing through the coronary vascular system, the perfusate flows through the coronary sinus and the opened right atrium, respectively. The cardiac cavities remain basically empty throughout the experiment. The primary reason for the investigation of a substance with an unknown effect in an isolated organ is the very independence of this isolated organ from nervous and humoral regulation as well as from the substrate supply by the complete organism.

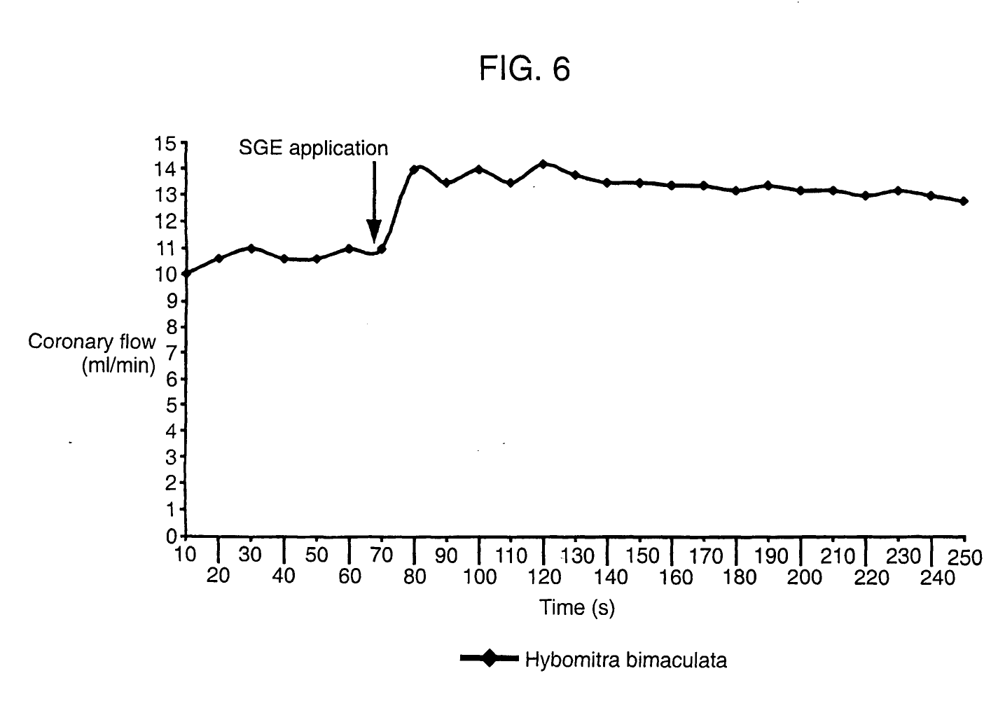

SGE from Hybomitra bimaculata that had displayed the maximal vasodilatory activities in rat femoral artery rings was selected for testing in the isolated perfused rat heart model (Langendorf constant pressure model). In this model, the Hybomitra bimaculata SGE increased coronary flow and left ventricular contractility, the most potent being SGE at the 100 μl dose. The results of this experiment are shown in Table 1 and FIG. 6.

TABLE 1
Effect of salivary gland extract from Hybomitra
bimaculata on the isolated perfused rat heart.

| | | Left | |
|---|---|---|---|
| | | Ventricular | Change in |
| Dose SGE (μl) | Coronary flow | Contractility | heart rate |
| 50 | +39% | +20% | 0 |
| 100 | +50% | +42.8% | −10% |
| 150 | +42% | +40% | +5% |

Purification and Identification of Active fractions from Hybomitra bimaculata SGE

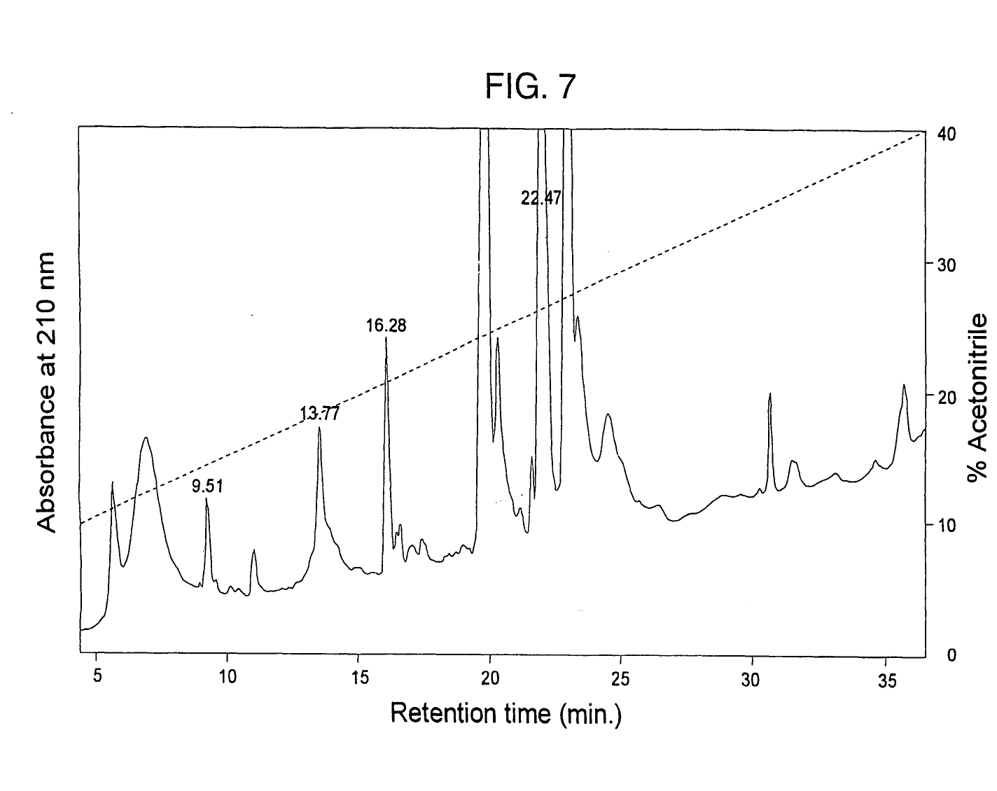

For the purification and identification of active fractions, salivary glands of Hybomitra bimaculata were used. FIG. 7 demonstrates the RP-HPLC chromatogram obtained from SGE of 475 pairs of salivary glands. A vasorelaxation activity of 47% was found in the peak with a retention time of 13.77 min; 45% relaxation was measured in the peak with a retention time of 16.28 min. Less activity (30%) was obtained with a peak of retention time 9.51 min, and 15% with a peak of retention time 22.47 min.

Amino Acid Analysis and Sequencing of Hybomitra bimaculata HPLC Fractions

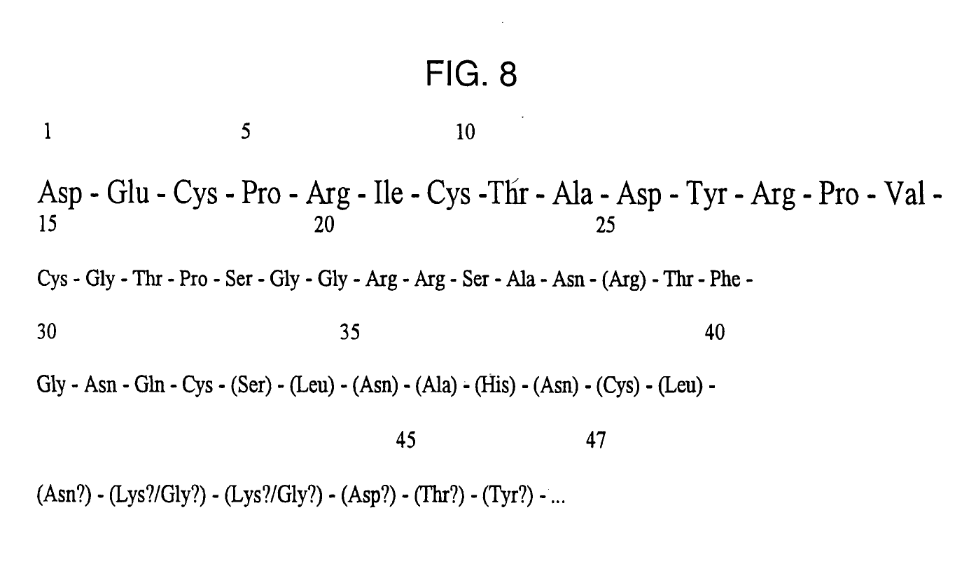

The HPLC fraction of retention time 14.51 min, which was derived from the 13.77 min peak (FIG. 7), was subjected to N-terminal Edman degradation and yielded a partial sequence of 47 amino acid residues (FIG. 8), designated EV048.

Primary Structure of the cDNA Encoding EV048

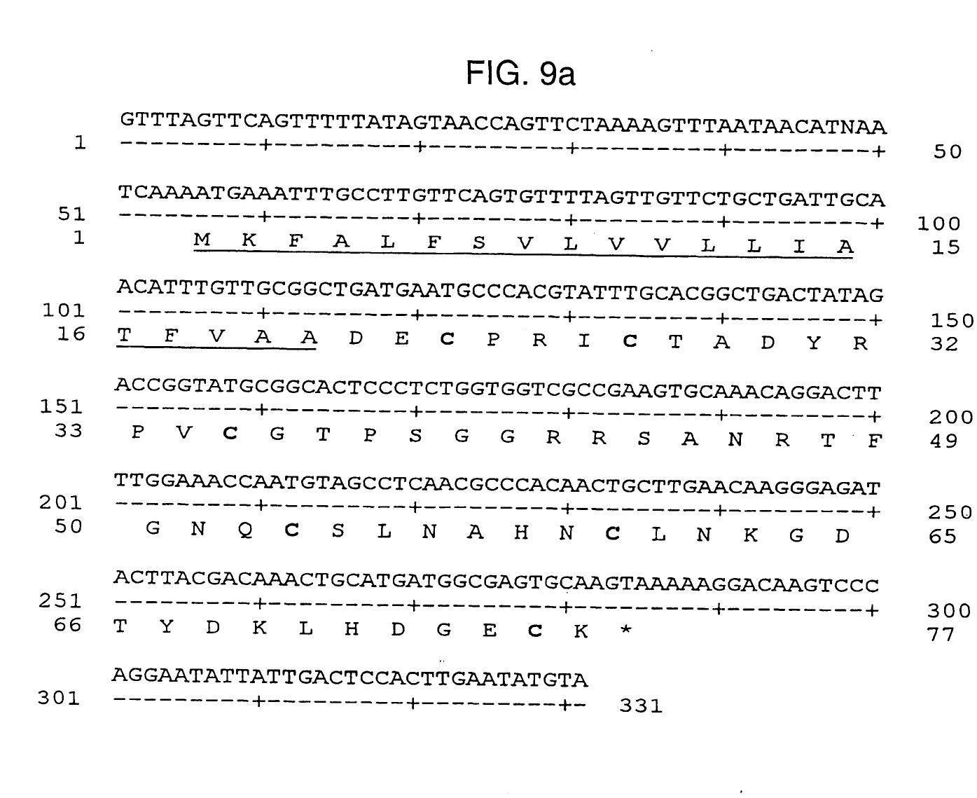

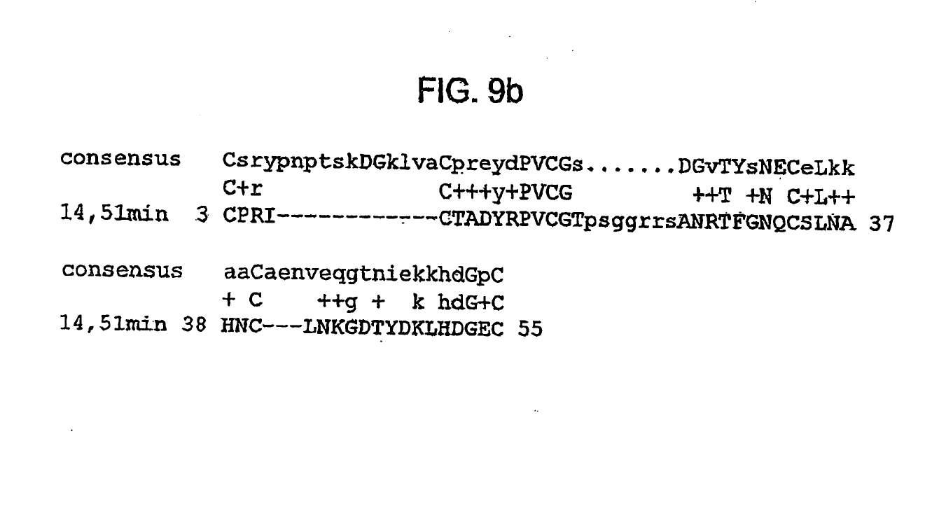

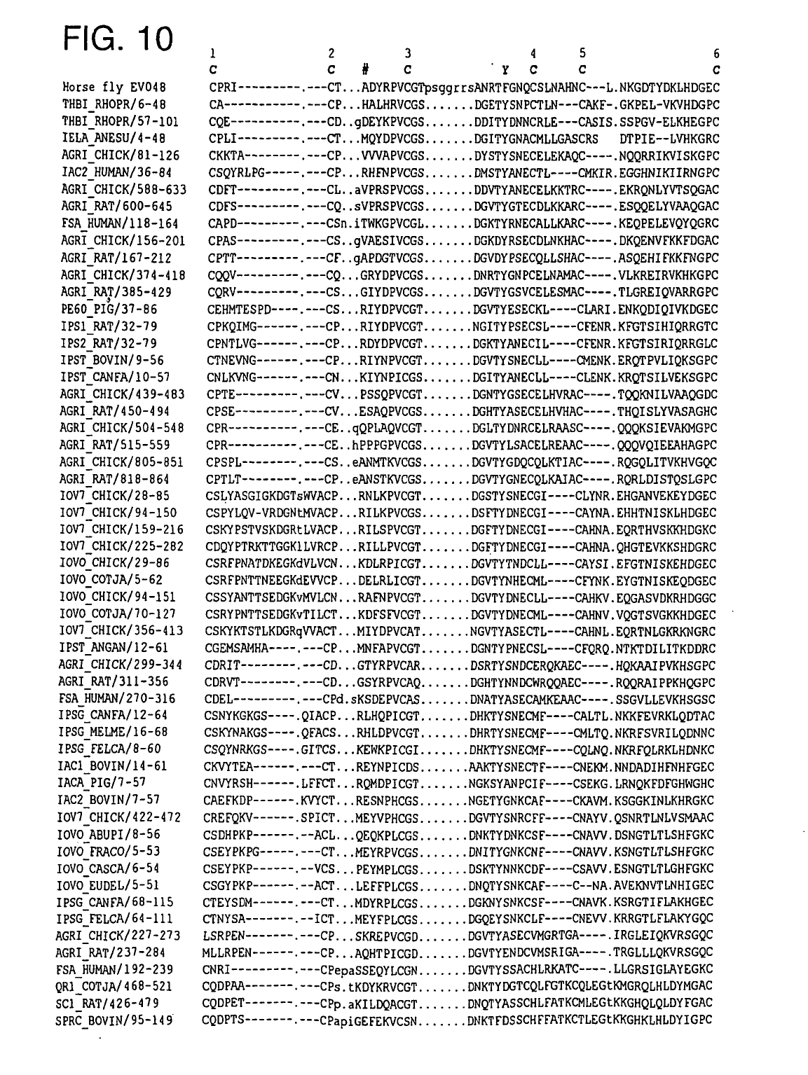

The full length cDNA for EV048 encodes a peptide of 76 amino acids (FIG. 9a). This includes a 20 amino acid putative signal peptide that is probably cleaved at VAA-DEC to generate the mature N-terminus (FIG. 8). The complete peptide has a predicted molecular weight of 8282.4 Da and a theoretical pI of 8.27. The 56 amino acid mature peptide has a predicted molecular weight of 6146.7 Da and a theoretical pI of 7.78. An N-linked glycosylation site is predicted at the asparagine residue at position 26 in the mature peptide; there are no predicted O-linked sites. The sequence of the mature peptide has similarity with Kazal-type protease inhibitors (FIG. 10), including homology with rhodniin I and II, the Kazal-type inhibitors from Rhodnius prolixus, another haematophagous insect species (Friedrich et al., 1993, van de Locht et al., 1995), and with the protease inhibitor from the sea anemone Anemonia sulcata that is specific for elastases (Tschesche et al., 1987).

The basic structure of a Kazal-type inhibitor is shown in the following schematic representation:

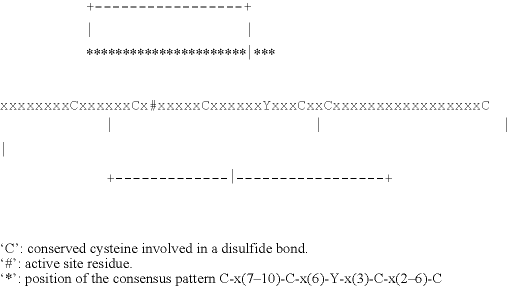

All six cysteines are conserved in EV048 although the spacing between cysteine residues within the consensus pattern is unusual C-x(7)-C-x(13)-F-x(3)-C-x(6)-C. This unusual pattern in EV048 is due to a sequence inserted between the third and fourth cysteines (PSGGRRS) that does not align with any other Kazal family member (FIG. 10). The second serine within the sequence has a high probability of phosphorylation but the rest of the seven amino acid sequence has no similarity to any other motifs in the PROSITE database. Homology modelling indicates that the insertion occurs within, or just before, the region of the peptide that forms the second β-sheet. The highly conserved tyrosine residue (shown in bold in FIG. 10) is a phenylalanine in EV048, and the putative active site residue (indicated by # in bold in FIG. 10) is an alanine.

Baculovirus Expression of EV048

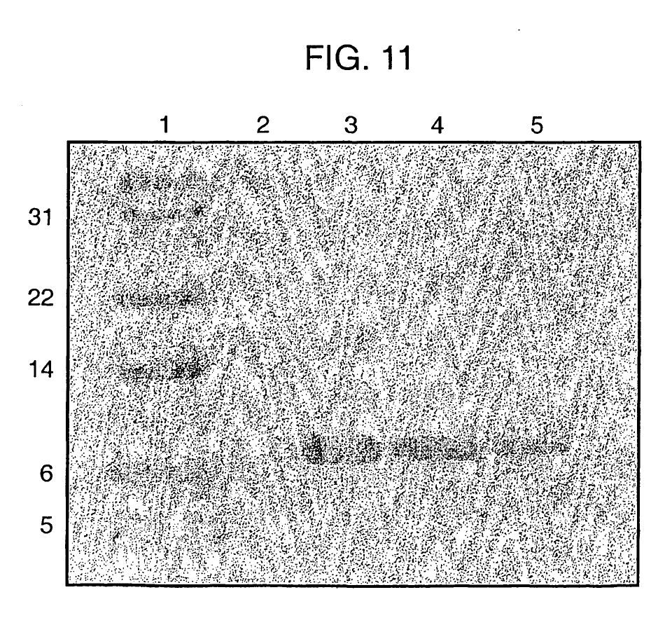

The expressed peptide is exported from the cell to the supernatant. Expression levels of EV048 in the supernatant were approximately 0.3% g per ml of Sf9 cells. The expressed protein is approximately the size (7 kD) expected for the mature peptide (with glycine and 6×HIS tag) and was purified to homogeneity (FIG. 11) in two steps.

Activity of EV048 on Isolated Rat Cardiomyocytes

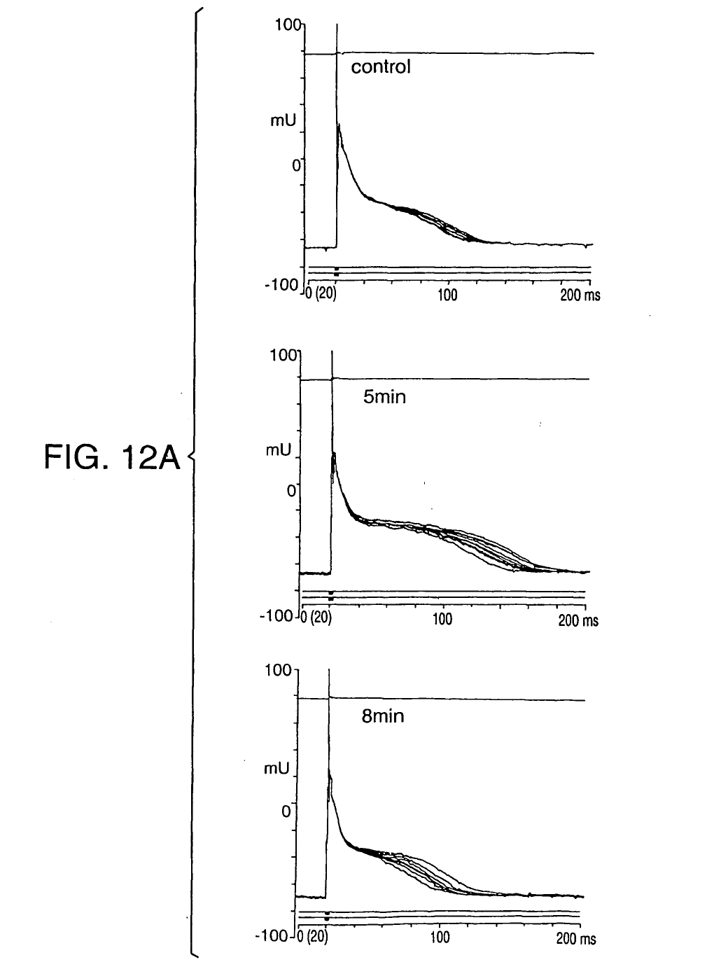

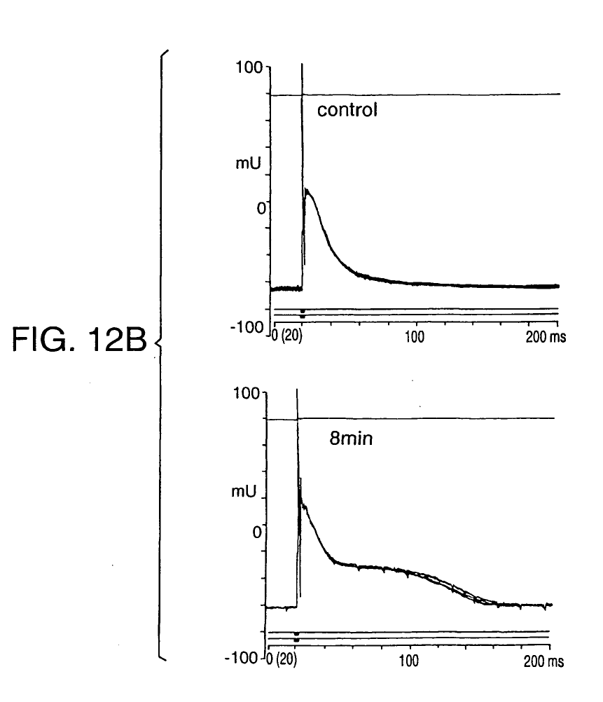

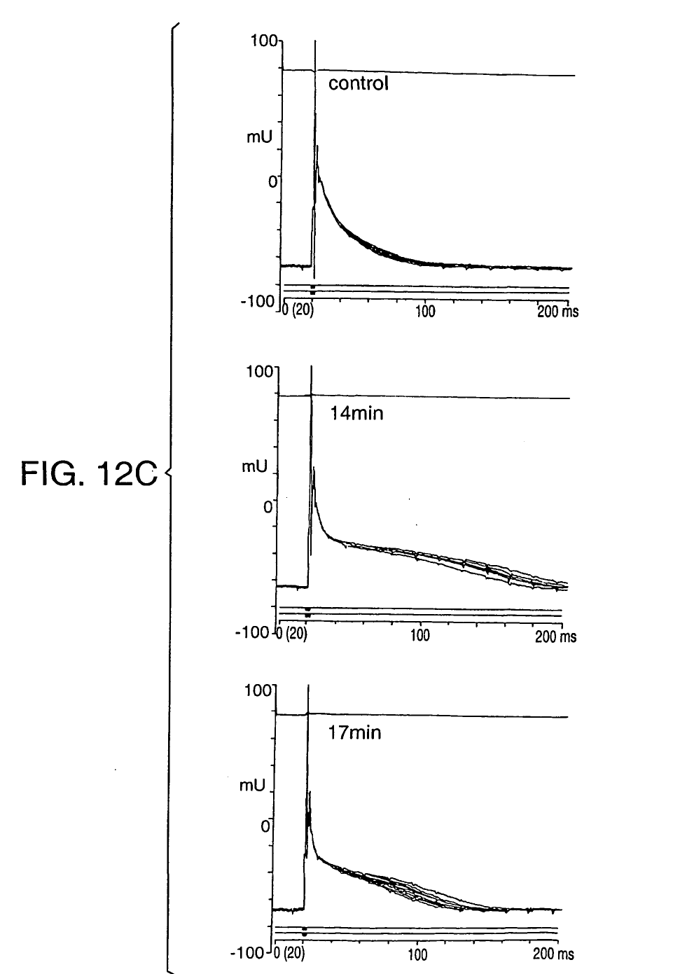

EV048, the recombinant peptide, derived from saliva of Hybomitra bimaculata (0.2 and 0.4 μg/ml) exhibited positive inotropism and prolongation of the action potential for the duration of exposure. These effects persisted for up to 16.5 minutes following washout. Spontaneous contractile activity was not observed at any time with this molecule. The results of a series of three experiments examining the effect of EV048 on the action potential of rat cardiomyocytes are shown in FIG. 12.

Effect of EV048 on the Isolated Perfused Rat Heart

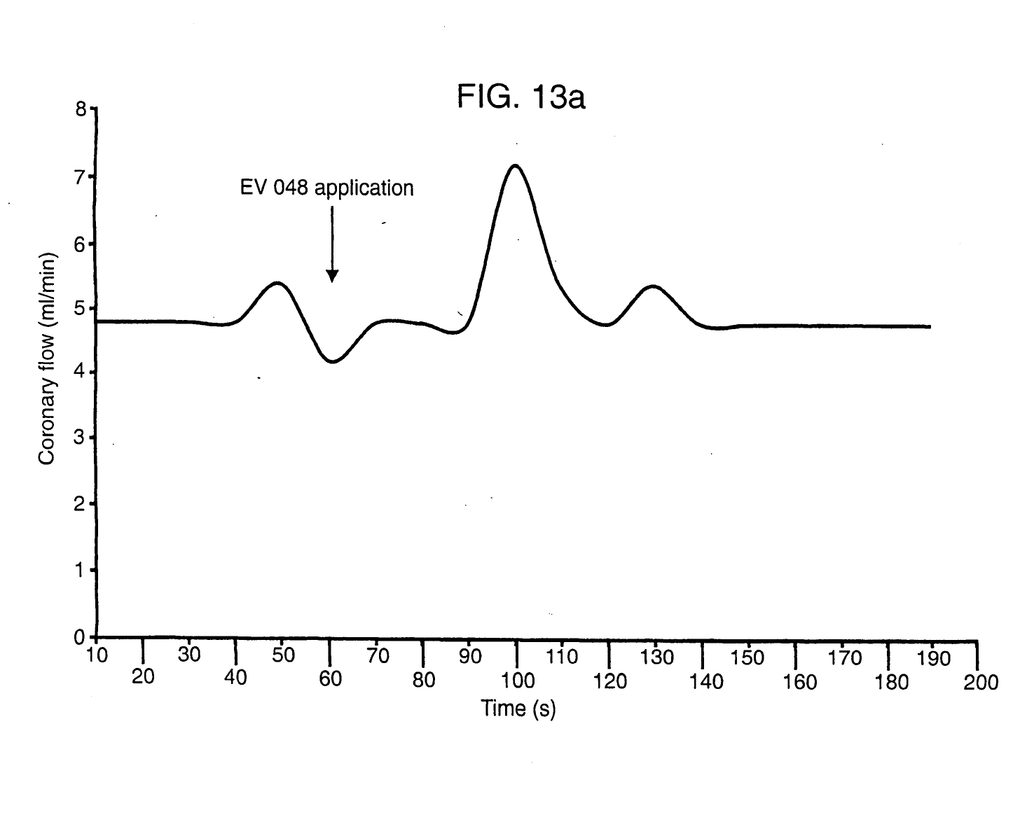

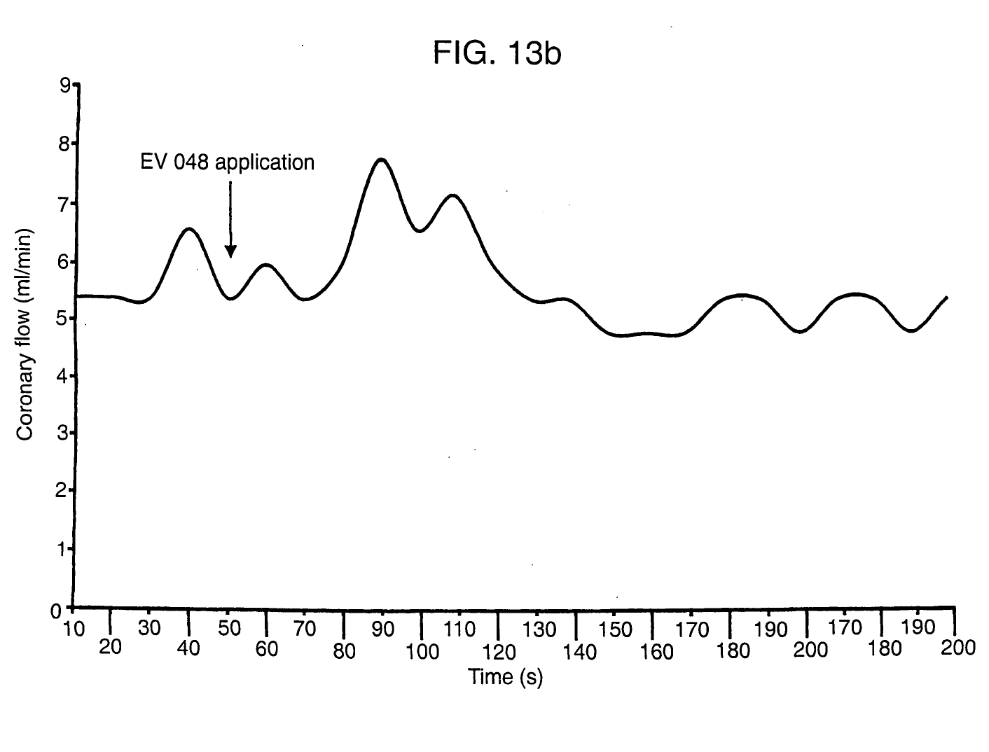

EV048 was tested in the isolated perfused rat heart model. This resulted in a transient increase in coronary blood flow with no alteration of heart rate or rhythm (FIGS. 13a and b).

Discussion

Vasodilatory Effects of Hybomitra bimaculata Salivary Gland Extract (SGE)

SGE from Hybomitra bimaculata induced relaxation of rat femoral artery exceeding 50%. Similar magnitude of arterial relaxation has been induced by SGE from several species of haematophagous insects including mosquitoes (Champagne and Ribeiro, 1994), black flies (Cupp et al., 1994), sand flies (Lerner and Shoemaker, 1992), ticks (Kemp et al., 1983) and triatomine bugs (Ribeiro et al., 1990, 1993). However the mode of action of the peptidic vasodilators found in the other species varies. None are ion channel modulators.

Tachykinins elicit release of nitric oxide following binding of the peptide to endothelial cell tachykinin receptors. Such binding induces endothelium-dependent vasorelaxation (Champagne and Ribeiro, 1994). By contrast, protein vasodilators such as nitrophorins are able to bind and release nitric oxide. Delivering NO to the host vessel induces direct relaxation of smooth muscle by increasing intracellular cGMP levels (Champagne, 1994; Weichsel et al., 1998). Other vasodilators like maxadilan increase the intracellular level of cAMP within smooth muscle cells leading to relaxation (Grevelink et al., 1995). Both vasodilating mechanisms are endothelium-independent. It is proposed that, unlike vasodilators found in other haematohagous arthropod species, EV048 does not act either as a tachykinin or as a NO donor.

Data presented here support endothelium-independent relaxation of rat femoral artery induced by Hybomitra bimaculata SGE. Indeed, SGE from this species induced higher levels of relaxation of the artery after endothelium removal. SGE from this horsefly species appears to act directly on smooth muscle cells, presumably by calcium channel blocking, and promote vasorelaxation without endothelium mediation, the endothelium representing, if anything, a barrier rather than part of the active process.

Effects on Sarcolemmal Na,K-ATPase:

The results clearly showed that the SGE from Hybomitra bimaculata contains at least one compound which, at low concentration, stimulates Na,K-ATPase but at higher concentration inhibits the enzyme. When 3 μg of SGE were applied it induced a significant stimulation of the Na,K-ATPase. Increasing the amount of SGE to 6.5 μg induced an inhibition of the Na,K-ATPase. This phenomenon may be fundamental to understanding the vasodilating activity of this SGE on the rat femoral artery, whilst also explaining the positive inotropism of the SGE from Hybomitra bimaculata in the isolated rat heart and of the recombinant molecule EV048 in isolated rat cardiomyocytes. Inhibition of Na,K-ATPase, as was found at higher concentrations of SGE, has long been known to be associated with positive inotropism as is the case with other inhibitors of such as ouabain (Allen et al. 1975) and vanadium (Schmitz et al. 1982).

Whilst the inhibitory effect on Na,K-ATPase of SGE from Hybomitra bimaculata may explain the positive inotropism of the same SGE in the isolated rat heart, it is difficult to explain the substantial relaxation of femoral artery smooth muscle when exposed to Hybomitra bimaculata SGE by the same mechanism and this, together with the increase in diastolic volume and the increase in coronary blood flow in the rat heart model, are more easily explained by calcium channel inhibition. However, the fact that stripping the vascular endothelium appears to enhance the degree of smooth muscle relaxation could also suggest the involvement of NO.

Cardioactive Effects of the Crude Hybomitra bimaculata SGE and a Recombinant Peptide Molecule, EV048, on the Isolated Perfused Rat Heart

The initial results of testing crude extracts of salivary glands from Hybomitra bimaculata in an isolated perfused rat heart model resulted in both increased coronary blood flow and left ventricular contractility. Investigation of EV048 in this model suggested that it has a positively inotropic effect, although limited availability of material meant that this effect was transient. No negative inotropism was seen with either the SGE or the recombinant molecule and there were no alterations in heart rate or rhythm. This combination of features in a potent vasodilator such as EV048 is unusual, if not unique, and endows it with substantial therapeutic potential.

Effects of a Recombinant Peptide, EV048, on Isolated Rat Cardiomyocytes

EV048 prolonged the action potential and showed positive inotropism in isolated rat cardiomyocytes without provoking spontaneous contractile activity. This combination of properties, together with the substantial increase in coronary blood flow caused by Hybomitra bimaculata SGE in the isolated perfused rat heart, is considered to give EV048 potential value as a therapeutic agent in situations where increased ventricular output is desirable without the risk of inducing arrhythmias. Such clinical situations might include haemorrhagic, cardiogenic and septic shock, intractable angina and heart failure following coronary thrombosis. The duration of action after removal of the active agent by washout (>16 minutes) is also considered to increase its potential value as a therapeutic agent. Prolongation of the action potential without apparently inducing spontaneous oscillations may also make it a useful anti-arrhythmic agent.

Kazal-Type Protein from Hybomitra bimaculata

Following protein fractionation of SGE from Hybomitra bimaculata by HPLC, the maximal vasodilating responses of the artery were induced by fractions with a retention time of 9.51, 13.77, 16.28, and 22.47 min (FIG. 6).

The amino acid analysis of the fraction with the maximum-inducing vasorelaxation (retention time 13.77 min), and subsequent analysis of the derived cDNA, indicated that the molecule designated EV048 is closely related to Kazal-type proteins. The Kazal family of proteins includes a variety of protease inhibitors including pancreatic secretory trypsin inhibitor (Greene and Giordano, 1969), avian ovomucoid (Laskowski et al, 1987), acrosin inhibitor (Williamson et al, 1984) and elastase inhibitor (Tschesche et al. 1987). Kazal inhibitors contain between 1 and 9 Kazal-type inhibitor repeats. Kazal protease inhibitors that inhibit trypsin-like proteinases have basic residues (R, K or H) at their active (or P1) site whereas those that inhibit chymotrypsin-like proteases have large hydrophobic residues at the P1 position. The putative active site residue of EV048 is the small hydrophobic amino acid alanine. This residue is not present in any other Kazal proteins known to inhibit proteases. However, the active site of EV048 appears similar to a modelled sequence predicted to have very tight binding to porcine pancreatic elastase (Lu et al, 2001).

The extra sequence (PSGGRRS) inserted between the third and fourth cysteine residues of EV048 may well play a role in the vasodilating properties of the peptide. Homology modelling suggests that the additional amino acids exist at an exposed location and may permit interaction with a target molecule. Initial studies with the recombinant molecule presented here have demonstrated biological activity consistent with the effects observed with crude SGE from Hybomitra bimaculata.

### REFERENCES

Akera T, Brody T M: in Human Pharmacology, Molecular to Clinical, Ed. Brody T M, Lamer J, Minneman K P, Mosby 1998; 213-226
Alberts B, Bray D, Johnson A, Lewis J, Raff M, Roberts K, Walter P: Essential Cell Biology; Garland Publishing Inc. 1998; 371-406
Allen J. C., Entman M. L. and Schwartz A. (1975) The nature of the transport adenosine triphosphatase-digitalis complex. VIII. The relationship between in vivo-formed (3-H-ouabain-Na⁺, K⁺-adenosine triphosphatase) complex and ouabain-induced positive inotropism. J Pharmacol Exp Ther 192:105-112.
Ausubel E. A. et al Current Protocols in Molecular Biology, Wiley Interscience, New York.
Camm A J in Clinical Medicine, Ed. Kumar P, Clark M, W B Saunders Co. Ltd. 1996a; 521-630;
Camm A J in Clinical Medicine, Ed. Kumar P, Clark M, W B Saunders Co. Ltd. 1996b; 574-575.
Champagne, D. E. (1994) The role of salivary vasodilators in bloodfeeding and parasite transmission. Parasitology Today 10, 430-433.
Champagne, D. E. and Ribeiro, J. M. C. (1994) Sialokinin I and II: Vasodilatory tachykinins from the yellow fever mosquito Aedes aegypti. Proceedings of the National Academy of Science U.S.A. 91, 138-142.
Cupp, M. S., Ribeiro, J. M. C. and Cupp, E. W. (1994) Vasodilative activity in black fly salivary glands. American Journal of Tropical Medicine and Hygiene 50, 241-246.
Fernandez J. M. & Hoeffler J. P., eds. (1998) Gene expression systems. Using nature for the art of expression. Academic Press, San Diego, London, Boston, New York, Sydney, Tokyo, Toronto.
Friedrich, T., Kroeger, B., Bialojan, S., Lemaire, H. G., Hoeffken, H. W., Reuschenbach, P., Otte, M., and Dodt, J. (1993) A Kazal-type inhibitor with thrombin specificity from Rhodnius prolixus. Journal of Biological Chemistry 268, 16216-16222.
Greene, L. J. and Giordano, J. S. J. (1969). The structure of the bovine pancreatic secretory trypsin inhibitor—Kazal's inhibitor. I. The isolation and amino acid sequences of the tryptic peptides from reduced aminoethylated inhibitor. Journal of Biological Chemistry 244, 285-298.
Grevelink, S. A., Osborne, J. Loscalzo, J. and Lerner, E. A. (1995) Vasorelaxant and second messenger effects of maxadilan. Journal of Pharmacology and Experimental Therapeutics 272, 33-37.Hille B. Ionic Channels of Excitable Membranes, 2^{nd} Edn., Simauer Associates, Sunderland Mass.
Hume J R, Woosley R L in Human Pharmacology, Molecular to Clinical, Ed. Brody T M, Larner J, Minneman K P, Mosby 1998; 195-212;
Kemp D H, Hales J R, Schleger A V, Fawcett A A. Comparison of cutaneous hyperemia in cattle elicited by larvae of Boophilus microplus and by prostaglandins and other mediators Experientia 1983 Jul. 15;39 (7):725-7
Langendorff, O. (1895) Untersuchungen am uberlebenden Saugetiereherzen. Pflugers Arch. ges. Physiol. 61, 291.
Laskowski M., Kato I., Ardelt W., Cook J., Denton A., Empie M. W., Kohr W. J., Park S. J., Parks K., Schatzley B. L., Schoenberger O. L., Tashiro M., Vichot G., Whatley H. E., Wieczorek A., Wieczorek M. (1987) Ovomucoid third domains from 100 avian species: isolation, sequences, and hypervariability of enzyme-inhibitor contact residues. Biochemistry 26, 202-221.
Lerner, E. A. and Shoemaker, C. B. (1992) Maxadilan: Cloning and functional expression of the gene encoding this potent vasodilator. Journal of Biological Chemistry 276, 1062-1066.
Locht, van de, A., Lamba, D., Bauer, M., Huber, R., Friedrich, T., Kroeger, B., Hoeffken, W., and Bode, W. (1995) Two heads are better than one: crystal structure of the insect derived double domain Kazal inhibitor rhodniin in complex with thrombin. EMBO Journal 14, 5149-5157.
Lowry, O. H., Rosebrough, N. J., Farr, A. L., and Randall, R. J. (1951) Protein measurement with the folin phenol reagent. J Biol Chem 193: 265-275.
Lu S M, Lu W, Qasim M A, Anderson S, Apostol I, Ardelt W, Bigler T, Chiang Y W, Cook J, James M N, Kato I, Kelly C, Kohr W, Komiyama T, Lin T Y, Ogawa M, Otlewski J, Park S J, Qasim S, Ranjbar M, Tashiro M, Warne N, Whatley H, Wieczorek A, Wieczorek M, Wilusz T, Wynn R, Zhang W, Laskowski M Jr (2001). Predicting the reactivity of proteins from their sequence alone: Kazal family of protein inhibitors of serine proteinases. Proc Natl Acad Sci USA, 98, 1410-5
Ribeiro, J. M. C., Marinotti, O. and Gonzales R. (1990) A salivary vasodilator in the blood sucking bug Rhodnius prolixus. British Journal of Pharmacology 101, 932-936.
Ribeiro, J. M. C., Hazzard, J. M. H., Nussenzveig, R. H., Champagne, D. E. and Walker, F. A. (1993) Reversible binding of nitric oxide by a salivary heme protein from a bloodsucking insect. Science 260, 539-541.
Sahai H, Ageel M I (2000) The Analysis of Variance. Boston, Birkhäuser.
Sambrook J. et al (1989) Molecular cloning: a laboratory manual New York: Cold Spring Harbour Laboratory Press.
Schmitz W., Scholz H., Erdmann E., Krawietz W. and Werdan K. (1982) Effect of vanadium in the +5, +4 and +3 oxidation states on cardiac force of contraction, adenylate cyclase and (Na⁺+K⁺)-ATPase activity. Biochem Pharmacol 23:3853-3860.
Spector et al (1998) Cells, a laboratory manual; Cold Spring Harbour Laboratory Press.
Taussky H. H. and Shorr E. E. (1953) A microcolorimetric method for the determination of inorganic phosphorous. J Biol Chem 202: 675-685.
Tschesche, H., Kolkenbrock, H. and Bode, W. (1987). The covalent structure of the elastase inhibitor from Anemonia sulcata—a “non-classical” Kazal type protein. 368, 1297-1304.
Vrbjar N, Soos J, and Ziegelhoffer A. (1984) Secondary structure of heart sarcolemmal proteins during interaction with metallic cofactors of (NA⁺+K⁺)-ATPase. Gen Physiol Biophys 3: 317-325.
Weichsel, A., Anderson, J. F., Champagne, D. E., Walker, F. A., Montfort, W. R. (1998) Crystal structure of a nitric oxide transport protein from a blood-sucking insect. Nature Structural Biology 5, 304-309.
Williamson M. P., Marion D., Wuthrich K. (1984) Secondary structure in the solution conformation of the proteinase inhibitor IIA from bull seminal plasma by nuclear magnetic resonance. Journal of Molecular Biology 173, 341-359.

1. An ion channel modulator molecule (ICMM) derived from a haematophagous arthropod, or a functional equivalent thereof.

2. An ICMM or functional equivalent according to claim 1, which modulates the activity of more than one ion channel simultaneously.

3. An ICMM or functional equivalent according to claim 1 wherein said ion channel(s) is selected from the group consisting of a calcium channel, a potassium channel, a sodium channel, and a sodium-potassium ATPase.

4. An ICMM or functional equivalent according to claim 1 that inhibits the activity of said ion channel(s).

5. An ICMM according to claim 4 that inhibits the activity of said ion channel(s) by binding to said ion channel(s).

6. An ICMM or functional equivalent according to claim 1 which is a vasodilator.

7. An ICMM or functional equivalent according to claim 6 wherein said vasodilation occurs through nitric oxide donation.

8. An ICMM or functional equivalent according to claim 6 which is a vasodilator of coronary vessels.

9. An ICMM or functional equivalent according to claim 6 which is a vasodilator of peripheral vessels.

10. An ICMM or functional equivalent according to claim 1 which does not have a negative inotropic effect.

11. An ICMM or functional equivalent according to claim 1 that has a positive inotropic effect.

12. An ICMM or functional equivalent according to claim 1 that prolongs the action potential of muscle cells.

13. An ICMM or functional equivalent according to claim 12 wherein said muscle cells are cardiomyocyte cells.

14. An ICMM or functional equivalent according to claim 1 which is derived from a haematophagous arthropod including all arthropods that take a blood meal from a suitable host such as insects, ticks, lice, fleas and mites.

15. An ICMM or functional equivalent according to claim 14 wherein said haematophagous arthropod is a horsefly of the Tabinadae family.

16. An ICMM or functional equivalent according to claim 15 wherein said horsefly is derived from the Hybomitra, Heptatoma, Chrysops, Haematopota or Tabanus genera.

17. An ICMM or functional equivalent according to claim 16 wherein said horsefly is Hybomitra bimaculata.

18. An ICMM or functional equivalent according to claim 1 comprising the sequence in FIG. 9a.

19. An ICMM or functional equivalent according to claim 18 which has greater than 50% identity with the sequence in FIG. 9a, preferably greater than 60%, 70%, 80%, 85%, 90%, 95%, 97%, 98% or 99% sequence identity, as defined using the GCG suite of programs (Wisconsin Package Version, 10.1, Genetics Computer Group (GCG), Madison, Wis.) or the ExPASy (ExpertProtein Analysis System) proteomics server of the Swiss Insitute of Bioinformatics.

20. An ICMM or functional equivalent according to claim 1, which is a recombinant protein.

21. An ICMM or functional equivalent according to claim 1, which is genetically or chemically fused to one or more peptides or polypeptides.

22. An ICMM or functional equivalent, according to claim 1, for use in therapy.

23. A nucleic acid molecule encoding an ICMM or functional equivalent according to claim 1.

24. A vector comprising a nucleic acid molecule according to claim 23.

25. A host cell transformed or transfected with a vector of claim 24.

26. A method of preparing an ICMM or functional equivalent, comprising introducing a vector according to claim 24 into a host cell and culturing said host cell under conditions wherein said ICMM or functional equivalent is expressed and recovering said ICMM or functional equivalent.

27. A method of isolating an ICMM or functional equivalent according to claim 1 comprising the steps of:
a) preparing an extract from a haematophagous arthropod,
b) separating said extract into fractions containing proteins,
c) testing said fractions for the ability to modulate ion channel activity
d) isolating said ICMM or functional equivalent from a fraction that possesses the ability to modulate said ion channel activity.

28. A method according to claim 27 wherein the extract is separated into fractions by fast phase or high performance liquid chromatography, ion exchange chromatography, affinity chromatography, gel filtration or reverse phase high performance liquid chromatography.

29. A method according to claim 27 wherein testing said fractions for the ability to modulate ion channel activity comprises testing for the ability to cause vasodilation and/or positive inotropism and/or lengthen of action potential.

30. A method according to claim 29 wherein testing for the ability to cause vasodilation comprises assessing the effect of the fractions on pre-contracted rat femoral artery rings or assessing the effect of fractions on coronary blood flow in an isolated Langendorf heart.

31. A method according to claim 29 wherein testing for the ability to cause positive inotropism comprises assessing the effect of fractions on whole cell patch clamping in isolated cardiomyocytes or assessing the effect of fraction on left ventricular output in an isolated perfused Langendorf heart.

32. A method according to claim 29 wherein testing for the ability to lengthen action potential comprises assessing the effect of fractions on whole cell patch clamping in isolated cardiomyocytes.

33. An ICMM or functional equivalent obtainable by the method of claim 27.

34. A method according to claim 27 comprising the additional steps of isolating and sequencing the gene encoding said ICMM or functional equivalent.

35. A method of isolating a gene encoding an ICMM or functional equivalent comprising performing the steps recited in claim 27, said method additionally comprising performing the steps of:
e) obtaining the N-terminal sequence of said isolated ICMM or functional equivalent;
f) designing a degenerate oligonucleotide; and
g) using said oligonucleotide to screen a library in order to isolate a gene encoding the ICMM or functional equivalent

36. A pharmaceutical composition comprising a material selected from the group consisting of an ICMM derived from a haematophagous arthropod, or functional equivalent thereof, and a nucleic acid molecule encoding said ICMM or functional equivalent thereof, in conjunction with a pharmaceutically acceptable carrier.

37. A method for the prevention or treatment of a disease or condition caused by a fault in ion channel activity comprising administering to a subject an effective dose of a material selected from the group consisting of an ICMM derived from a haematophagous arthropod, or functional equivalent thereof, a nucleic acid molecule encoding said ICMM or functional equivalent thereof, and a composition according to claim 36.

38. A method according to claim 37 wherein said disease is selected from cardiac conditions such as coronary insufficiency leading to angina, congestive cardiac failure and cardiac arrhythmias; peripheral vascular disease such as cerebro-vascular insufficiency, intermittent claudication and Buerger's disease; vasospastic disorders such as Raynaud's disease, cerebral or coronary vasospasm; reperfusion following stroke and myocardial infarction; shock including septic shock, haemorrhagic shock and cardiogenic shock; hypertension; to assist in circulatory support during and following cardio-pulmonary by-pass or angioplasty procedures.

39. A process for the formulation of a composition according to claim 36 comprising bringing said ICMM or functional equivalent, or said nucleic acid molecule, into association with a pharmaceutically acceptable carrier or adjuvant.

40. A method for studying the effect of ion channel modulation, including vasodilation, inotropism and lengthening of action potential, in vitro comprising administering to a cell or an organ an ICMM or functional equivalent according to claim 1.

41. An ion channel modulator comprising a polypeptide or peptide having the sequence psggrrs.

42. An ion channel modulator comprising a Kazal type protein.

43. The ion channel modulator of either of claims 41 or 42, wherein said ion channel is selected from the group comprising a sodium channel, a potassium channel, a calcium channel or a sodium-potassium ATPase.

44. The ion channel modulator of either of claims 41 or 42, wherein said ion channel modulator is a vasodilator.

45. A method for treating or preventing a disease or condition caused by a fault in ion channel activity, comprising administering a therapeutically effective amount of a medicament, said medicament comprising a polypeptide or peptide having the sequence psggrrs, and a Kazal type protein.

46. A pharmaceutical composition comprising a peptide or polypeptide as recited in claim 42 in combination with a pharmaceutically acceptable carrier.

47. A method for studying ion channel modulation in vitro comprising administering to a cell or an organ a peptide, polypeptide or Kazal type protein as recited in either of claims 41 or 42.
